SEQUENCE LISTING (23 sequences)

SEQ ID NO: 1 - protein, 5 aa, synthetic construct
SYWMY

SEQ ID NO: 2 - protein, 17 aa, synthetic construct
HINPSSGYTKYNQKFKD

SEQ ID NO: 3 - protein, 7 aa, synthetic construct
DPNWDNY

SEQ ID NO: 4 - protein, 11 aa, synthetic construct
KASQDVSIAVA

SEQ ID NO: 5 - protein, 7 aa, synthetic construct
SASYRYT

SEQ ID NO: 6 - protein, 9 aa, synthetic construct
QQHYSTPYT

SEQ ID NO: 7 - protein, 116 aa, synthetic construct
QVQLVQSGAEVKKPGASVKVSCKASGYTFTSYWMYWVRQAPGQGLEWIGHINPSSGYTKYNQKFKDRATLTADTSTSTAYMELSSLRSEDTAVYYCARDPNWDNYWGQGTTVTVSS

SEQ ID NO: 8 - protein, 116 aa, synthetic construct
QVQLVQSGAEVKKPGASVKVSCKASGYTFTSYWMYWVRQAPGQGLEWIGHINPSSGYTKYNQKFKDRVTMTADTSTSTVYMELSSLRSEDTAVYYCARDPNWDNYWGQGTTVTVSS

SEQ ID NO: 9 - protein, 116 aa, synthetic construct
QVQLQQSGAEVKKPGASVKLSCKASGYTFTSYWMYWVRQRPGQGLEWIGHINPSSGYTKYNQKFKDRATLTADTSTSTAYMELSSLRSEDTAVYYCARDPNWDNYWGQGTTVTVSS

SEQ ID NO: 10 - protein, 116 aa, synthetic construct
QVQLVQSGAEVKKPGASVKVSCKASGYTFTSYWMYWVRQAPGQGLEWIGHINPSSGYTKYNQKFKDKATLTADKSTSTAYMELSSLRSEDTAVYYCARDPNWDNYWGQGTTVTVSS

SEQ ID NO: 11 - protein, 116 aa, synthetic construct
QVQLVQSGAEVKKPGASVKVSCKASGYTFTSYWMYWVRQAPGQGLEWIGHINPSSGYTKYNQKFKDKATLTADKSTSTAYMELSSLRSEDTAVYYCARDPNWDNYWGQGTTLTVSS

SEQ ID NO: 12 - protein, 116 aa, synthetic construct
QVQLQQSGAELAKPGASVKLSCKASGYTFTSYWMYWVKQRPGQGLEWIGHINPSSGYTKYNQKFKDKATLTADKSSSTAYMQLSSLTYEDSAVYYCARDPNWDNYWGQGTTLTVSS

SEQ ID NO: 13 - protein, 107 aa, synthetic construct
DIVMTQSPSSLSASVGDRVSITCKASQDVSIAVAWYQQKPGKAPKLLIYSASYRYTGVPDRFSGSGSGTDFTFTISSLQPEDIATYYCQQHYSTPYTFGGGTKLEIK

SEQ ID NO: 14 - protein, 107 aa, synthetic construct
DIQMTQSPSSLSASVGDRVTITCKASQDVSIAVAWYQQKPGKAPKLLIYSASYRYTGVPSRFSGSGSGTDFTFTISSLQPEDIATYYCQQHYSTPYTFGGGTKLEIK

SEQ ID NO: 15 - protein, 107 aa, synthetic construct
DIQMTQSPSSMSASVGDRVTITCKASQDVSIAVAWYQQKPGKAPKLLIYSASYRYTGVPSRFSGSGSGTDFTFTISSVQPEDIATYYCQQHYSTPYTFGGGTKLEIK

SEQ ID NO: 16 - protein, 107 aa, synthetic construct
DIVMTQSHKFMSTSVGDRVSITCKASQDVSIAVAWYQQKPGQSPKLLIYSASYRYTGVPDRFTGSGSGTDFTFTISSVQAEDLAVYYCQQHYSTPYTFGGGTKLEIK

SEQ ID NO: 17 - protein, 121 aa, synthetic construct
QVQLQQWGAGLLKPSETLSLTCAVYGGSFSGYYWSWIRQPPGKGLEWIGEINHSGSTNYNPSLKSRVTISVDTSKNQFSLKLSSVTAADTAVYYCARLPTRWVTTRYFDLWGRGTLVTVSS

SEQ ID NO: 18 - protein, 111 aa, synthetic construct
QSVLTQPPSVSGAPGQRVTISCTGSSSNIGAGYDVHWYQQLPGTAPKLLIYGNSNRPSGVPDRFSVSKSGASASLAITGLQAEDEADYYCQSFDNSLSAYVFGGGTQLTVL

SEQ ID NO: 19 - protein, 123 aa, synthetic construct
QVTLKESGPGILQPSHTLSLTCSFSGFSLNTYAMGVSWIRQPSGKGLEWLASIWWNGNKYNNPSLKSRLTVSKDTSNNQAFLKVTSVDTADTATYYCAHSRIIRFTDYVMDAWGQGASVTVSS

SEQ ID NO: 20 - protein, 107 aa, synthetic construct
DIQMTQSPASLSTFLGEPVTIECRASEDIYNDLAWYQQKPGKSPQLLIYNANSLHTGVPSRFSGSGSGTQYSLKINSLQSEDVASYFCQQYYDYPLTFGSGTKLEIK

SEQ ID NO: 21 - protein, 12 aa, synthetic construct
NGQFHIPSITWE

SEQ ID NO: 22 - protein, 18 aa, synthetic construct
LYREKKSASWITRIRPEL

SEQ ID NO: 23 - protein, 597 aa, Homo sapiens
MTPIVTVLICLGLSLGPRTRVQTGTIPKPTLWAEPDSVITQGSPVTLSCQGSLEAQEYRLYREKKSASWITRIRPELVKNGQFHIPSITWEHTGRYGCQYYSRARWSELSDPLVLVMTGAYPKPTLSAQPSPVVTSGGRVTLQCESQVAFGGFILCKEGEDEHPQCLNSQPHARGSSRAIFSVGPVSPNRRWSHRCYGYDLNSPYVWSSPSDLLELLVPGVSKKPSLSVQPGPVMAPGESLTLQCVSDVGYDRFVLYKEGERDLRQLPGRQPQAGLSQANFTLGPVSRSYGGQYRCYGAHNLSSECSAPSDPLDILITGQIRGTPFISVQPGPTVASGENVTLLCQSWRQFHTFLLTKAGAADAPLRLRSIHEYPKYQAEFPMSPVTSAHAGTYRCYGSLNSDPYLLSHPSEPLELVVSGPSMGSSPPPTGPISTPGPEDQPLTPTGSDPQSGLGRHLGVVIGILVAVVLLLLLLLLLFLILRHRRQGKHWTSTQRKADFQHPAGAVGPEPTDRGLQWRSSPAADAQEENLYAAVKDTQPEDGVEMDTRAAASEAPQDVTYAQLHSLTLRRKATEPPPSQEREPPAEPSIYATLAIH

(19) United States
(12) Patent Application Publication
(10) Pub. No.: US 2026/0008847 A1
(43) Pub. Date: Jan. 8, 2026

(54) ANTI-LILRB2 ANTIBODIES AND USES THEREOF

(71) Applicants: ELPISCIENCE (SUZHOU) BIOPHARMA, LTD., SUZHOU (CN); ELPISCIENCE BIOPHARMA, LTD., SHANGHAI (CN)

(72) Inventors: Xiaofeng NIU, SHANGHAI (CN); Chunnian WANG, SHANGHAI (CN); Jinfeng ZHAO, SHANGHAI (CN); Ying WANG, SHANGHAI (CN); Yanfen HU, SHANGHAI (CN); Jun SUN, SHANGHAI (CN); Quan QIU, SHANGHAI (CN); Zhihao WU, SHANGHAI (CN); Yangsheng QIU, SHANGHAI (CN); Hongtao LU, SHANGHAI (CN)

(73) Assignees: ELPISCIENCE (SUZHOU) BIOPHARMA, LTD., SUZHOU, JS (CN); ELPISCIENCE BIOPHARMA, LTD., SHANGHAI (CN)

(21) Appl. No.: 18/992,162

(22) PCT Filed: Jul. 7, 2023
(86) PCT No.: PCT/CN2023/106392

§ 371 Date: Jan. 7, 2025

(30) Foreign Application Priority Data
Jul. 8, 2022 (WO) PCT/CN2022/104494
Jun. 29, 2023 (CN) 202310787466X

(51) Int. Cl. C07K 16/28; A61P 37/04

(52) U.S. Cl. CPC C07K 16/2803; A61P 37/04; C07K 16/2818; C07K 2317/24; C07K 2317/34; C07K 2317/565; C07K 2317/76; C07K 2317/92

(57) ABSTRACT

The present disclosure relates to an antibody or antigen-binding fragment thereof that binds to LILRB2 (Leukocyte Immunoglobulin Like Receptor B2), the anti-LILRB2 antibody or antigen-binding fragment thereof could block the signaling pathway and upregulates the immune response to a LILRB2-related disease, disorder, or condition.

### CROSS-REFERENCE TO RELATED APPLICATIONS

This application claims priority to PCT International Application No. PCT/CN2022/104494, filed on Jul. 8, 2022, and Chinese Patent Application No. 202310787466.X, filed on Jun. 29, 2023. The disclosure of the prior application is considered part of the disclosure of this application and is incorporated in its entirety into this application.

### REFERENCE TO THE SEQUENCE LISTING

The Sequence Listing titled DCF230536WO-Seqlisting.xml, which was created on Jul. 6, 2023 and is 23,061 bytes in size, is hereby incorporated by reference in its entirety.

### TECHNICAL FIELD

The present disclosure relates to an antibody or antigen-binding fragment thereof that binds to LILRB2 (Leukocyte Immunoglobulin Like Receptor B2) and uses thereof.

### BACKGROUND

The statements in this section merely provide background information related to the present disclosure and do not necessarily constitute prior art.

Members of the leukocyte immunoglobulin-like receptor (LILR) family are critical positive (LILRA) and negative (LILRB) regulators of innate and adaptive immune responses. LILRB family members show distinct expression patterns on immune cells with LILRB2 (Leukocyte Immunoglobulin Like Receptor B2, UniProt: A2IXV5) being mainly restricted to myeloid origin cells, including monocytes, macrophages, dendritic cells, and granulocytes. LILRB2, also named monocyte/macrophage immunoglobulin-like receptor 10 (MIR-10), immunoglobulin-like transcript 4 (ILT4), or CD85 antigen-like family member D (CD85D), is a classic type I transmembrane protein with four extracellular tandem Ig-like domains, a transmembrane region, and a cytoplasmic tail with 3 immunoreceptor tyrosine inhibitory motifs (ITIMs) (Aiqin Gao et al., ILT4 functions as a potential checkpoint molecule for tumor immunotherapy. Reviews on Cancer. 1869 (2018) 278-285).

Interactions between LILRB2 and specific ligands are required for the molecular processes with unique biological functions. Human LILRB2 has originally been shown to broadly bind MHC-I molecules, also known as human leukocyte antigen (HLA) class I molecules (HLA-A, HLA-B, HLA-C) and non-classical HLA-class I (HLA-E, HLA-F, HLA-G, and HLA-H). Subsequent studies demonstrate LILRB2 binding to other non-HLA ligands including angiopoietin-like (ANGPTL) family, myelin-associated glycoprotein (MAG), and oligomeric beta-amyloid (William et al., LILRB receptor-mediated regulation of myeloid cell maturation and function. Cancer Immunol Immunother. 2017; 66(8): 1079-1087).

LILRB2 is a potent inhibitory molecule with immune suppressive functions. Recent studies have shown that LILRB2 is overexpressed or highly induced in tumor microenvironment as well as in malignant tumor cells from both hematopoietic and solid tumors. Clinically, expression levels of LILRB2 in tumor cells of NSCLC and breast cancer patients are positively correlated with the poor cell differentiation, increased regional lymph node metastasis, advanced cancer stages and poor patient survival, (Aiqin Gao et al., ILT4 functions as a potential checkpoint molecule for tumor immunotherapy. Reviews on Cancer. 1869 (2018) 278-285). Accumulating evidence highlights the potential role of LILRB2 as a novel immune checkpoint for tumor immunotherapy.

Immunotherapy has become a breakthrough strategy to treat human cancer, but there remains a need for more effective treatments of cancer utilizing immunotherapy. More particularly, there remains a need for novel anti-LILRB2 antibodies, compositions and methods containing the same.

### SUMMARY

For the above-mentioned purpose, provided herein is a novel antibody or antigen-binding fragment thereof that binds to LILRB2, and the uses thereof.

In one aspect, the disclosure provides an antibody or antigen-binding fragment thereof. The antibody or antigen-binding fragment thereof comprises a heavy chain variable region (VH) comprising complementarity determining regions (CDRs) 1, 2, and 3, wherein the VH CDR1 comprises an amino acid sequence that is at least 80%, 85%, 90%, 95%, 96%, 97%, 98%, 99% identical to the sequence set forth in SEQ ID NO: 1; the VH CDR2 comprises an amino acid sequence that is at least 80%, 85%, 90%, 95%, 96%, 97%, 98%, 99% identical to the sequence set forth in SEQ ID NO: 2; the VH CDR3 comprises an amino acid sequence that is at least 80%, 85%, 90%, 95%, 96%, 97%, 98%, 99% identical to the sequence set forth in SEQ ID NO: 3; and a light chain variable region (VL) comprising CDRs 1, 2, and 3, wherein the VL CDR1 comprises an amino acid sequence that is at least 80%, 85%, 90%, 95%, 96%, 97%, 98%, 99% identical to the sequence set forth in SEQ ID NO: 4; the VL CDR2 comprises an amino acid sequence that is at least 80%, 85%, 90%, 95%, 96%, 97%, 98%, 99% identical to the sequence set forth in SEQ ID NO: 5; the VL CDR3 comprises an amino acid sequence that is at least 80%, 85%, 90%, 95%, 96%, 97%, 98%, 99% identical to the sequence set forth in SEQ ID NO: 6.

In some embodiments, the VH of the antibody or the antigen-binding fragment thereof comprises CDRs 1, 2, and 3 with the amino acid sequences set forth in SEQ ID NOs: 1, 2, 3, respectively; and the VL comprises CDRs 1, 2, and 3 with the amino acid sequences set forth in SEQ ID NOs: 4, 5, 6, respectively.

In some embodiments, the VH of the antibody or the antigen-binding fragment thereof is at least 80%, 85%, 86%, 87%, 88%, 89%, 90%, 91%, 92%, 93%, 94%, 95%, 96%, 97%, 98%, 99% identical to the amino acid sequence set forth in SEQ ID NO: 7, 8, 9, 10, 11 or 12; and the VL is at least 80%, 85%, 86%, 87%, 88%, 89%, 90%, 91%, 92%, 93%, 94%, 95%, 96%, 97%, 98%, 99% identical to the amino acid sequence set forth in SEQ ID NO: 13, 14, 15 or 16.

In some embodiments, the VH of the antibody or the antigen-binding fragment thereof is different from the amino acid sequence set forth in SEQ ID NO: 7, 8, 9, 10, 11 or 12 by no more than 25, 20, 15, 13, 10, 9, 8, 7, 6, 5, 4, 3, 2 or 1 amino acid; the VL is different from the amino acid sequence set forth in SEQ ID NO: 13, 14, 15 or 16 by no more than 20, 15, 13, 10, 9, 8, 7, 6, 5, 4, 3, 2 or 1 amino acid.

In some embodiments, the VH of the antibody or antigen-binding fragment thereof comprises the amino acid sequence set forth in SEQ ID NO: 7, 8, 9, 10, 11 or 12; and the VL comprises the amino acid sequence set forth in SEQ ID NO: 13, 14, 15 or 16.

In some embodiments, the VH comprises the amino acid sequence of SEQ ID NO: 7 and the VL comprises the amino acid sequence of SEQ ID NO: 13; the VH comprises the amino acid sequence of SEQ ID NO: 8 and the VL comprises the amino acid sequence of SEQ ID NO: 13; the VH comprises the amino acid sequence of SEQ ID NO: 9 and the VL comprises the amino acid sequence of SEQ ID NO: 13; the VH comprises the amino acid sequence of SEQ ID NO: 7 and the VL comprises the amino acid sequence of SEQ ID NO: 14; the VH comprises the amino acid sequence of SEQ ID NO: 8 and the VL comprises the amino acid sequence of SEQ ID NO: 14; the VH comprises the amino acid sequence of SEQ ID NO: 9 and the VL comprises the amino acid sequence of SEQ ID NO: 14; the VH comprises the amino acid sequence of SEQ ID NO: 10 and the VL comprises the amino acid sequence of SEQ ID NO: 15; or the VH comprises the amino acid sequence of SEQ ID NO: 11 and the VL comprises the amino acid sequence of SEQ ID NO: 15; the VH comprises the amino acid sequence of SEQ ID NO: 12 and the VL comprises the amino acid sequence of SEQ ID NO: 16.

In some embodiments, the antibody or antigen-binding fragment thereof specifically binds to LILRB2. In some embodiments, the antibody or antigen-binding fragment thereof specifically binds to LILRB2 with an EC₅₀ value of less than 20 nM, less than 10 nM, less than 5 nM or less than 1 nM.

In some embodiments, the antibody or antigen-binding fragment thereof blocks the interaction between human LILRB2 and HLA-A2 (major histocompatibility complex, class I, A2).

In some embodiments, the antibody or antigen-binding fragment thereof blocks the interaction between human LILRB2 and HLA-A2 with an IC₅₀ value of less than 20 nM, less than 10 nM, less than 5 nM, less than 1 nM, or less than 0.6 nM as measured by FACS (Fluorescence Activated Cell Sorting) assay.

In some embodiments, the antibody or antigen-binding fragment thereof blocks the interaction between human LILRB2 and HLA-G (major histocompatibility complex, class I, G).

In some embodiments, the antibody or antigen-binding fragment thereof blocks the interaction between human LILRB2 and HLA-G with an IC₅₀ value of less than 10 nM, less than 5 nM, less than 1 nM, less than 0.5 nM, or less than 0.3 nM as measured by FACS (Fluorescence Activated Cell Sorting) assay.

In some embodiments, the antibody or antigen-binding fragment thereof blocks the interaction between human LILRB2 and human ANGPTL1 (Angiopoietin-like 1).

In some embodiments, the antibody or antigen-binding fragment thereof blocks the interaction between human LILRB2 and human ANGPTL2 (Angiopoietin-like 2).

In some embodiments, the antibody or antigen-binding fragment thereof blocks the interaction between human LILRB2 and human ANGPTL4 (Angiopoietin-like 4).

In some embodiments, the antibody or antigen-binding fragment thereof blocks the interaction between human LILRB2 and human ANGPTL7 (Angiopoietin-like 7).

In some embodiments, the antibody or antigen-binding fragment thereof blocks the interaction between human LILRB2 and human MAG (myelin associated glycoprotein).

In some embodiments, the antibody or antigen-binding fragment thereof is capable to reprogram human monocyte derived M2 macrophages into M1 phenotype (pro-inflammation M1 phenotype).

In some embodiments, the antibody or antigen-binding fragment thereof is capable to relieve M2 macrophages mediated suppression of T cells.

In some embodiments, the antibody or antigen-binding fragment thereof is capable to promote human dendritic cells into a maturation status.

In some embodiments, the antibody or antigen-binding fragment thereof is capable to promote human monocytes into a pro-inflammation status.

In some embodiments, the antibody or antigen-binding fragment thereof is capable to promote human PBMCs (Peripheral blood mononuclear cells) into a pro-inflammation status. In some embodiments, the antibody or antigen-binding fragment thereof is capable to enhance the expression of pro-inflammatory cytokines and/or chemokines. In some embodiments, the antibody or antigen-binding fragment thereof is capable to upregulate the release of pro-inflammatory cytokines and/or chemokines. In some embodiments, the pro-inflammatory cytokines comprise IL-2 (interleukin 2), IFN-γ (interferon gamma) and/or TNF-α (tumor necrosis factor). In some embodiments, the pro-inflammatory cytokines and/or chemokines are selected from the group consisting of IL-2, IFN-γ, TNF-α, Granzyme B, PDGF-AA, IL-3, IL-1beta, IL-5, TGF-alpha, IFN-alpha, CCL4, FGF-basic, IL-13, G-CSF, IL-4, PD-L1, CCL20, IL-15, GM-CSF, CCL5, CCL3, PDGF-AB, CCL19, CXCL2, IL-6, VEGF, and the combination thereof.

In some embodiments, the antibody or antigen-binding fragment thereof is capable to reduce the expression of IL-10 (interleukin 10).

In some embodiments, the antibody or antigen-binding fragment thereof binds human LILRB2 with KD (affinity constant) of less than 15 nM, less than 10 nM, less than 8 nM, or less than 6 nM as measured by FACS (Fluorescence Activated Cell Sorting) assay.

In some embodiments, the antibody or antigen-binding fragment thereof is a chimeric or humanized antibody or antigen-binding fragment thereof.

In some embodiments, the antibody or antigen-binding fragment thereof is an IgG1 antibody, IgG2 antibody, IgG3 antibody, or IgG4 antibody or antigen-binding fragment thereof.

In one aspect, provided herein is an antibody or antigen-binding fragment thereof which binds to the peptide including amino acid sequence set forth in SEQ ID NO: 21.

In one aspect, provided herein is an isolated polynucleotide encoding the antibody or antigen-binding fragment thereof that binds to LILRB2.

In one aspect, provided herein is an isolated vector comprising the polynucleotide encoding the antibody or antigen-binding fragment thereof that binds to LILRB2.

In one aspect, provided herein is a host cell comprising the isolated polynucleotide encoding the antibody or antigen-binding fragment thereof that binds to LILRB2 or the isolated vector comprising the polynucleotide described above.

In one aspect, provided herein is a pharmaceutical composition comprising the antibody or antigen-binding fragment thereof that binds to LILRB2 or the isolated polynucleotide encoding the antibody or antigen-binding fragment thereof that binds to LILRB2, or the isolated vector provided herein, and a pharmaceutically acceptable carrier.

In one aspect, the disclosure provides a pharmaceutical composition comprising the antibody or antigen-binding fragment thereof provided above and an anti-PD-1 antibody (e.g., Keytruda).

In one aspect, provided herein is a kit comprising the antibody or antigen-binding fragment thereof that binds to LILRB2 or the isolated polynucleotide encoding the antibody or antigen-binding fragment thereof that binds to LILRB2, or the isolated vector provided herein, or the host cell provided herein, or the pharmaceutical composition provided herein.

In one aspect, provided herein is the use of the antibody or antigen-binding fragment thereof or the isolated polynucleotide or the isolated vector or the host cell or the pharmaceutical composition or the kit provided herein in the manufacture of a therapeutic agent for binding to human LILRB2.

In one aspect, provided herein is the use of the antibody or antigen-binding fragment thereof binding LILRB2 or the isolated polynucleotide or the isolated vector or the host cell or the pharmaceutical composition or the kit in the manufacture of a therapeutic agent for blocking the interaction between human LILRB2 and HLA-A2, HLA-G, human ANGPTL1, human ANGPTL2, human ANGPTL4, human ANGPTL7 and/or human MAG.

In one aspect, provided herein is the use of the antibody or antigen-binding fragment thereof that binds to LILRB2 or the isolated polynucleotide or the isolated vector or the host cell or the pharmaceutical composition or the kit provided herein in the manufacture of a therapeutic agent for enhancing immune response.

In one aspect, provided herein is the use of the antibody or antigen-binding fragment thereof that binds to LILRB2, or the isolated polynucleotide provided herein, or the isolated vector provided herein, or the host cell provided herein, or the pharmaceutical composition provided herein, or the kit provided herein in the manufacture of a therapeutic agent for diagnosing, preventing or treating a disease.

In one aspect, provided herein is a method of enhancing immune response in a subject in need thereof, the method comprising administrating to the subject a therapeutically effective amount of the antibody or antigen-binding fragment thereof that binds to LILRB2, or the isolated polynucleotide provided herein, or the isolated vector provided herein, or the host cell provided herein, or the pharmaceutical composition provided herein, or the kit provided herein.

In one aspect, provided herein is a method of diagnosing or preventing or treating a disease, disorder or condition in a subject in need thereof, comprising administrating to the subject a therapeutically effective amount of the antibody or antigen-binding fragment thereof that binds to LILRB2, or the isolated polynucleotide provided herein, or the isolated vector provided herein, or the host cell provided herein, or the pharmaceutical composition provided herein, or the kit provided herein.

In some embodiments, the disease, disorder or condition comprises a tumor, optionally, at least a tumor cell expresses LILRB2.

In some embodiments, the subject is mammals including human.

In one aspect, provided herein is a binding epitope on LILRB2, wherein the binding epitope comprises the amino acid sequence set forth in SEQ ID NO: 21. In some embodiments, the LILRB2 is human LILRB2.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 shows the binding of 043c, 1E1 and J-19 to LILRB2 ECD recombinant protein as measured by ELISA. The mean OD450 values obtained with the indicated antibodies are listed at the top of each column. OD450 is the abbreviation for optical density at 450 nm. hIgG4 iso represents a human IgG4 isotype control.

FIG. 2 shows the binding of 043c to EL4 (EL4 murine tumor cell line)-LILRB2 cells as measured by FACS. MFI is the abbreviation for mean fluorescence intensity, and Ab conc. is the abbreviation for concentration of antibody (μg/ml). hIgG4 iso represents a human IgG4 isotype control.

FIG. 3 shows the binding of 043c and 1E1 to human primary monocytes isolated from healthy donor #1045 (FIG. 3A) and #6004 (FIG. 3B). MFI is the abbreviation for mean fluorescence intensity, and Ab conc. is the abbreviation for concentration of antibody (μg/ml). hIgG4 iso represents a human IgG4 isotype control.

FIG. 4 shows the binding of 043c, 1E1 and J-19 to LILRA1 (FIG. 4A), LILRA2 (FIG. 4B), LILRA3 (FIG. 4C), LILRA4 (FIG. 4D), LILRA5 (FIG. 4E), LILRB1 (FIG. 4F), LILRB3 (FIG. 4G), LILRB4 (FIG. 4H), LILRB5 (FIG. 4I) ECD recombinant proteins as measured by ELISA. The mean OD450 values obtained with the indicated antibodies are listed at the top of each column. OD450 is the abbreviation for optical density at 450 nm. hIgG4 iso represents a human IgG4 isotype control, and PC represents a positive control.

FIG. 5 shows the activity of 043c, 1E1 and J-19 to block the interaction between LILRB2 and HLA-A2, HLA-G, ANGPTL1, ANGPTL2, ANGPTL4, ANGPTL7, or MAG as measured by FACS based competition assay. The inhibition ratios obtained with the indicated antibodies are listed at the top of each column. hIgG4 iso represents a human IgG4 isotype control.

FIG. 6 shows the activity of 043c, 1E1 and J-19 to block the interaction between LILRB2 and HLA-G as measured by FACS based competition assay. The IC₅₀ values obtained with the indicated antibodies are listed at the right of each curve legend. Ab conc. is the abbreviation for concentration of antibody (μg/ml).

FIG. 7 shows the activity of 043c to block the interaction between LILRB2 and HLA-A2 as measured by FACS based competition assay. The IC₅₀ value obtained with 043c is listed at the right of the curve legend. Ab conc. is the abbreviation for concentration of antibody (μg/ml).

FIG. 8 shows the effects of 043c, 1E1 and J-19 to promote human PBMCs into a pro-inflammation status. TNFα (FIG. 8A) and IL10 (FIG. 8B) secretion from LPS (Lipopolysaccharide) primed human PBMCs under treatment of the indicated antibodies were detected by HTRF (Fluorescence Resonance Energy Transfer). The cytokine level values are listed at the top of each column. hIgG4 iso represents a human IgG4 isotype control, and Medium represents a negative control without any antibody treatment.

FIG. 9 shows the effects of 043c, 1E1 and J-19 to promote human monocytes into a pro-inflammation status. TNFα (FIG. 9A) and IL10 (FIG. 9B) secretion from LPS (Lipopolysaccharide) primed human primary monocytes under treatment of the indicated antibodies were detected by HTRF (Fluorescence Resonance Energy Transfer). The cytokine level values are listed at the top of each column. hIgG4 iso represents a human IgG4 isotype control, and Medium represents a negative control without any antibody treatment.

FIG. 10 shows the effects of 043c, 1E1 and J-19 to reprogram human monocyte derived M2 macrophages into pro-inflammation M1 phenotype. The M1 macrophage population switch (CD86^{high}CD163^{low}, FIG. 10A) and TNFα secretion (FIG. 10B) under treatment of the indicated antibodies were detected by FACS or HTRF (Fluorescence Resonance Energy Transfer), respectively. The values of M1 macrophage population percentage and TNFα level are listed at the top of each column. hIgG4 iso represents a human IgG4 isotype control, Medium represents a negative control without any antibody treatment, and M1 Control represents a positive control using monocyte derived M1 macrophages.

FIG. 11 shows the effects of 043c, 1E1 and J-19 to relieve M2 macrophages mediated suppression of T cells. IL-2 (FIG. 11A), IFN γ (FIG. 11B) and TNFα (FIG. 11C) secretion under treatment of the indicated antibodies were detected by HTRF (Fluorescence Resonance Energy Transfer). The cytokine level values are listed at the top of each column. M2:T=1:0.5, M2:T=1:1, M2:T=1:2, M2:T=1:4, and M2:T=1:8 represent that M2 macrophages were mixed with autologous T cells at various ratios. hIgG4 iso represents a human IgG4 isotype control, and Medium represents a negative control without any antibody treatment.

FIG. 12 shows the effects of 043c, 1E1 and J-19 to promote human monocyte derived dendritic cells into a maturation status. The CD86^{high}CD163^{low} dendritic cell population switch under treatment of the indicated antibodies was detected by FACS. The population percentage values are listed at the top of each column. hIgG4 iso represents a human IgG4 isotype control, and Medium represents a negative control without any antibody treatment.

FIG. 13 shows the binding epitopes of 043c (FIG. 13A) and 1E1 (FIG. 13B) on LILRB2 detected by HDX-MS (Hydrogen-deuterium exchange mass spectrometry). HDX deuteration % is the abbreviation for the percentage of hydrogen-deuterium exchange.

FIG. 14 shows the binding of 043c and 043c derived humanized variants (043c, hu043.01, hu043.02, hu043.03, hu043.04, hu043.05 and hu043.06) to EL4 (EL4 murine tumor cell line)-LILRB2 cells. The EC₅₀ values obtained with the indicated antibodies are listed at the right of each curve legend. MFI is the abbreviation for the mean fluorescence intensity, and Ab conc. is the abbreviation for the concentration of antibody (μg/ml). hIgG4 iso represents a human IgG4 isotype control.

FIG. 15 shows the activity of 043c, hu043.01, hu043.02, hu043.03, hu043.04, hu043.05, hu043.06, J-19 (FIG. 15A) and hu043.07, hu043.08 (FIG. 15B) to block the interaction between LILRB2 and HLA-G as measured by FACS based competition assay. The IC₅₀ values obtained with the indicated antibodies are listed at the right of each curve legend. Ab conc. is the abbreviation for the concentration of antibody (μg/ml). hIgG4 iso represents a human IgG4 isotype control.

FIG. 16 shows the effects of hu043.07, hu043.08, 043c, 1E1 and J-19 to promote human monocytes into a pro-inflammation status. TNFα secretion from LPS (Lipopolysaccharide) primed human primary monocytes under treatment of the indicated antibodies were detected by HTRF (Fluorescence Resonance Energy Transfer). The cytokine level values are listed at the top of each column. hIgG4 iso represents a human IgG4 isotype control, and Medium represents a negative control without any antibody treatment.

FIG. 17 shows the effects of hu043.07, hu043.08, 043c, 1E1 and J-19 to reprogram human monocyte derived M2 macrophages into pro-inflammation M1 phenotype. The M1 macrophage population switch (CD86^{high}CD163^{low}, FIG. 17A) and TNFα secretion (FIG. 17B) under treatment of the indicated antibodies were detected by FACS or HTRF (Fluorescence Resonance Energy Transfer), respectively. The values of M1 macrophage population percentage and TNFα level are listed at the top of each column. hIgG4 iso represents a human IgG4 isotype control, Medium represents a negative control without any antibody treatment, and M1 Control represents a positive control using monocyte derived M1 macrophages.

FIG. 18 shows the effect of hu043.08 in monotherapy and in combination with Keytruda on relieving M2 macrophages mediated suppression of T cells. IL-2 (FIG. 18A), IFNγ (FIG. 18B) and TNFα (FIG. 18C) secretion under the indicated treatments were detected by HTRF. The cytokine level values are listed at the top of each column. hIgG4 iso represents a human IgG4 isotype control, and Medium represents a negative control without any antibody treatment.

### DETAILED DESCRIPTION

The present disclosure is explained in greater detail below. This description is not intended to be a detailed catalog of all the different ways in which the invention may be implemented, or all the features that may be added to the instant invention. For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which do not depart from the instant invention. Hence, the following description is intended to illustrate some certain embodiments of the invention, and not to exhaustively specify all permutations, combinations and variations thereof.

Unless defined otherwise, all technical and scientific terms used herein have the same meaning as commonly understood by one of ordinary skill in the art to which the disclosure pertains. Although any methods and materials similar or equivalent to those described herein may be used in the practice for testing of the present disclosure, the preferred materials and methods are described herein. In describing and claiming the present disclosure, the following terminology will be used.

LILRB2 (Leukocyte Immunoglobulin Like Receptor B2, also known as MIR-10, ILT4 or CD85D) is a cell-surface receptor which can negatively regulate the immune response by recruiting tyrosine phosphatases, like SHP1 or SHP2. LILRB2 is predominantly expressed in monocytes, macrophages, and myeloid cells and be expressed by endothelial cells, placental trophoblasts, and decidual macrophages, suggesting that LILRB2 may play an important role in various physiological functions (Jian He, et al., Overexpression of ANGPTL2 and LILRB2 as predictive and therapeutic biomarkers for metastasis and prognosis in colorectal cancer. Int J Clin Exp Pathol. 2018; 11(5): 2281-2294).

LILRB2 functions to suppress myeloid cell activation, antigen presentation, and immune response. ILT4 is also highly expressed in various tumor cells and stroma cells, such as myeloid-derived suppressor cells (MDSCs) and tumor-associated macrophages (TAMs) in the TME (tumor microenvironment), directly modulating tumor growth, progression and metastasis (Aiqin Gao, et al., Tumor-derived ILT4 induces T cell senescence and suppresses tumor immunity. J Immunother Cancer. 2021; 9(3): e001536).

LILRB2 is a potential checkpoint molecule for tumor immunotherapy. LILRB2 blockade can promote antitumor immunity.

The present disclosure provides examples of antibodies and antigen-binding fragments thereof, that bind to LILRB2, and the uses of these antibodies and antigen-binding fragments thereof in the manufacture of a therapeutic agent (e.g., a drug) for binding to LILRB2.

### Definition

As used herein, the term “antibody” herein is used in the broadest sense and encompasses various antibody structures, including but not limited to monoclonal antibodies, polyclonal antibodies, multi-specific antibodies (for example, bispecific antibody), and antibody fragments so long as they exhibit the desired antigen-binding activity.

As used herein, the term “antigen-binding fragment” refers to a portion of a full-length antibody, wherein the portion of the antibody is capable of specifically binding to an antigen. In some embodiments, the antigen-binding fragment contains at least one variable domain (e.g., a variable domain of a heavy chain or a variable domain of light chain).

As used herein, the term “humanized antibody” refers to a non-human antibody which contains minimal sequence derived from a non-human (e.g., mouse) immunoglobulin and contains sequences derived from a human immunoglobulin.

As used herein, the term “subject” refers to human and non-human animals. Non-human animals include all mammals, such as mice, rats, rabbits, cats, dogs, pig, monkey, chimpanzee, gorilla, and the like. Except when noted, the term “patient” or “subject” are used herein interchangeably.

As used herein, the term “polynucleotide” refers to polymers of nucleotides of any length of at least two nucleotides, and include, without limitation, DNA, RNA, DNA/RNA hybrids, and modifications thereof.

As used herein, the term “vector” refers to a polynucleotide that can be engineered to contain a cloned polynucleotide or polynucleotides that can be propagated in a host cell. A vector can include one or more of the following elements: an origin of replication, one or more regulatory sequences (such as, for example, promoters and/or enhancers) that regulate the expression of the polypeptide of interest, and/or one or more selectable marker genes (such as, for example, antibiotic resistance genes and genes that can be used in colorimetric assays, for example, β-galactosidase).

As used herein, the term “host cell” refers to a cell that may be or has been a recipient of a vector or isolated polynucleotide. Host cells may be prokaryotic cells or eukaryotic cells. Exemplary eukaryotic cells include mammalian cells, such as primate or non-primate animal cells.

As used herein, the term “IL-2” is interleukin 2, the term “IL-2” and “IL2” are interchangeably used herein. The term “IL-3” is interleukin 3, the term “IL-3” and “IL3” are interchangeably used herein. The term “IL-4” is interleukin 4, the term “IL-4” and “IL4” are interchangeably used herein. The term “IL-5” is interleukin 5, “IL-5” and “IL5” are interchangeably used herein. The term “IL-6” is interleukin 6, the term “IL-6” and “IL6” are interchangeably used herein. The term “IL-8” is interleukin 8, the term “IL-8” and “IL8” are interchangeably used herein. The term “IL-10” is interleukin 10, the term “IL-10” and “IL10” are interchangeably used herein. The term “IL-13” is interleukin 13, the term “IL-13” and “IL13” are interchangeably used herein. The term “IL-15” is interleukin 15, the term “IL-15” and “IL15” are interchangeably used herein. The term “IL-1beta” or the term “IL-1F2” is interleukin 1 beta, the term “IL-1beta”, “IL-1β”, “IL-1F2”, “IL1beta”, “IL1β” and “IL1F2” are interchangeably used herein. The term “IL-1alpha” or the term “IL-1Fa” is interleukin 1 alpha, the term “IL-1alpha”, “IL-1Fa”, “IL-1α”, “IL1alpha”, “IL1Fa” and “IL1α” are interchangeably used herein.

As used herein, the term “IFNγ” is interferon gamma. The term “IFN-alpha” is interferon alpha 1, the term “IFN-alpha”, “IFN-α”, “IFN alpha” and “IFNα” are interchangeably used herein. The term “IFN-beta” is interferon beta, the term “IFN-beta”, “IFN-β”, “IFN beta”, “IFNβ” are interchangeably used herein.

As used herein, the term “TNFα” is tumor necrosis factor, the term “TNFα” and “TNF-alpha” are interchangeably used herein.

As used herein, the term “PDGF-AA” is platelet derived growth factor subunit A alpha, the term “PDGF-AB” is platelet derived growth factor subunit A beta, and the term “PDGF-BB” is platelet derived growth factor subunit B beta.

As used herein, the term “TGF-alpha” is transforming growth factor alpha, the term “TGFα” and “TGF-alpha” are interchangeably used herein.

As used herein, the term “CCL2” or the term “MIP-1” or the term “JE” is C—C motif chemokine ligand 2, the term “CCL3” or the term “MIP-1 alpha” is C—C motif chemokine ligand 3, the term “CCL4” or the term “MIP-1 beta” is C—C motif chemokine ligand 4, the term “CCL5” is C—C motif chemokine ligand 5, the term “CCL19” or the term “MIP-3 beta” is C—C motif chemokine ligand 19, the term “CCL20” or the term “MIP-3 alpha” is C—C motif chemokine ligand 20, the term “MIP-3” is C—C motif chemokine ligand 23, the term “RANTES” is C—C motif chemokine ligand 5, the term “CCL22” or the term “MDC” is C—C motif chemokine ligand 22, and the term “CCL23” or the term “MPIF-1” is C—C motif chemokine ligand 23.

As used herein, the terms “FGF-basic” is fibroblast growth factor 2.

As used herein, the terms “G-CSF” is colony stimulating factor 3, and the terms “GM-CSF” is colony stimulating factor 2.

As used herein, the terms “B7-H1” or the terms “PD-L1” is programmed cell death 1 ligand 1.

As used herein, the terms “CXCL2” or the term “GRO beta” is C—X—C motif chemokine ligand 2, and the terms “CXCL8” is C—X—C motif chemokine ligand 8.

As used herein, the terms “VEGF” is vascular endothelial growth factor.

### Antibodies and Antigen-Binding Fragments Thereof

Provided herein is an antibody or antigen-binding fragment thereof that binds to LILRB2, or an anti-LILRB2 antibody or antigen-binding fragment thereof.

In general, antibodies are made up of two classes of polypeptide chains, light chains and heavy chains. An antibody can comprise two identical copies of a light chain and two identical copies of a heavy chain. The heavy chains, which each contain one variable region (or variable domain, VH) and multiple constant regions (or constant domains), bind to one another via disulfide bonding within their constant domains. The light chains, which each contain one variable region (or variable domain, VL) and one constant region (or constant domain), each bind to one heavy chain via disulfide binding. The variable regions of both the light chains and heavy chains contain three hypervariable regions sandwiched between more conserved framework regions (FR).

These hypervariable regions, known as the complementary determining regions (CDRs), form loops that comprise the principal antigen binding surface of the antibody. The CDRs are important for recognizing an epitope of an antigen. As used herein, an “epitope” is the smallest portion of a target molecule capable of being specifically bound by the antigen binding domain of an antibody.

In some embodiments, the antibody or the antigen-binding fragment thereof comprising:
a heavy chain variable region (VH) comprising complementarity determining regions (CDRs) 1, 2, and 3, wherein the VH CDR1 comprises an amino acid sequence that is at least 80%, 85%, 90%, 95%, 96%, 97%, 98%, 99% identical to the sequence set forth in SEQ ID NO: 1; the VH CDR2 comprises an amino acid sequence that is at least 80%, 85%, 90%, 95%, 96%, 97%, 98%, 99% identical to the sequence set forth in SEQ ID NO: 2; the VH CDR3 comprises an amino acid sequence that is at least 80%, 85%, 90%, 95%, 96%, 97%, 98%, 99% identical to the sequence set forth in SEQ ID NO: 3; and
a light chain variable region (VL) comprising CDRs 1, 2, and 3, wherein the VL CDR1 comprises an amino acid sequence that is at least 80%, 85%, 90%, 95%, 96%, 97%, 98%, 99% identical to the sequence set forth in SEQ ID NO: 4; the VL CDR2 comprises an amino acid sequence that is at least 80%, 85%, 90%, 95%, 96%, 97%, 98%, 99% identical to the sequence set forth in SEQ ID NO: 5; the VL CDR3 comprises an amino acid sequence that is at least 80%, 85%, 90%, 95%, 96%, 97%, 98%, 99% identical to the sequence set forth in SEQ ID NO: 6.

CDRs are defined using Kabat definition. The Kabat definition is well known to those skilled in the art, see, for example, Kabat, E. A. et al. Sequences of Proteins of Immunological Interest, 1991.

In some embodiments, the antibody or the antigen-binding fragment thereof can have a VH comprising CDRs 1, 2, and 3, wherein the CDRs 1, 2, and 3 are set forth in SEQ ID NOs: 1, 2, 3, respectively.

Furthermore, the antibodies or the antigen-binding fragments thereof can have a VL comprising CDRs 1, 2, and 3, wherein the CDRs 1, 2, and 3 are set forth in SEQ ID NOs: 4, 5, 6, respectively.

In some embodiments, the antibody or the antigen-binding fragment thereof have a VH comprising CDRH1 (heavy chain CDR1) of SEQ ID NO: 1, CDRH2 (heavy chain CDR2) of SEQ ID NO: 2, CDRH3 (heavy chain CDR3) of SEQ ID NO: 3.

In some embodiments, the antibody or the antigen-binding fragment thereof have a VL comprising CDRL1 (light chain CDR1) of SEQ ID NO: 4, CDRL2 (light chain CDR2) of SEQ ID NO: 5, CDRL3 (light chain CDR3) of SEQ ID NO: 6.

In some embodiments, the antibody or antigen-binding fragment thereof can have a VH, wherein the VH is at least 80%, 85%, 86%, 87%, 88%, 89%, 90%, 91%, 92%, 93%, 94%, 95%, 96%, 97%, 98%, 99% identical to the amino acid sequence set forth in SEQ ID NO: 7, 8, 9, 10, 11 or 12.

In some embodiments, the antibody or antigen-binding fragment thereof can have a VL, wherein the VL is at least 80%, 85%, 86%, 87%, 88%, 89%, 90%, 91%, 92%, 93%, 94%, 95%, 96%, 97%, 98%, 99% identical to the amino acid sequence set forth in SEQ ID NO: 13, 14, 15 or 16.

The method regarding how to determine the identity percentage of two amino acid sequences is known in the art, include but not limited to, BLAST (Basic Local Alignment Search Tool) on NCBI.

In some embodiments, the antibody or antigen-binding fragment thereof can have a VH, wherein the VH is different from the amino acid sequence set forth in SEQ ID NO: 7, 8, 9, 10, 11 or 12 by no more than 25, 20, 15, 13, 10, 9, 8, 7, 6, 5, 4, 3, 2 or 1 amino acid.

Furthermore, in some embodiments, the antibody or antigen-binding fragment thereof can have a VL, wherein the VL is different from the amino acid sequence set forth in SEQ ID NO: 13, 14, 15 or 16 by no more than 20, 15, 13, 10, 9, 8, 7, 6, 5, 4, 3, 2 or 1 amino acid.

In some embodiments, the antibody or antigen-binding fragment thereof comprises a VH selected from the group of SEQ ID NOs: 7, 8, 9, 10 and 11; and/or a VL selected from the group of SEQ ID NO: 13, 14 and 15.

Any of the VH sequences (SEQ ID NOs: 7-11) provided by present disclosure can be paired with any of the VL sequences (SEQ ID NOs: 13-15) provided by present disclosure.

In some embodiments, the antibody or antigen-binding fragment thereof described herein can have a VH of the SEQ ID NO: 7 with 0, 1, 2, 3, 4, 5, 6, 7, 8, 9, or 10 amino acid insertions, deletions or substitutions; SEQ ID NO: 8 with 0, 1, 2, 3, 4, 5, 6, 7, 8, 9, or 10 amino acid insertions, deletions or substitutions; SEQ ID NO: 9 with 0, 1, 2, 3, 4, 5, 6, 7, 8, 9, or 10 amino acid insertions, deletions or substitutions; SEQ ID NO: 10 with 0, 1, 2, 3, 4, 5, 6, 7, 8, 9, or 10 amino acid insertions, deletions or substitutions; or SEQ ID NO: 11 with 0, 1, 2, 3, 4, 5, 6, 7, 8, 9, or 10 amino acid insertions, deletions or substitutions.

In some embodiments, the antibody or antigen-binding fragment thereof described herein can have a VL of the SEQ ID NO: 13 with 0, 1, 2, 3, 4, 5, 6, 7, 8, 9, or 10 amino acid insertions, deletions, or substitutions; SEQ ID NO: 14 with 0, 1, 2, 3, 4, 5, 6, 7, 8, 9, or 10 amino acid insertions, deletions or substitutions; or SEQ ID NO: 15 with 0, 1, 2, 3, 4, 5, 6, 7, 8, 9, or 10 amino acid insertions, deletions or substitutions.

In some embodiments, provided herein is an antibody or antigen-binding fragment thereof, wherein the VH comprises the amino acid sequence of SEQ ID NO: 7 and the VL comprises the amino acid sequence of SEQ ID NO: 13,
the VH comprises the amino acid sequence of SEQ ID NO: 8 and the VL comprises the amino acid sequence of SEQ ID NO: 13,
the VH comprises the amino acid sequence of SEQ ID NO: 9 and the VL comprises the amino acid sequence of SEQ ID NO: 13,
the VH comprises the amino acid sequence of SEQ ID NO: 7 and the VL comprises the amino acid sequence of SEQ ID NO: 14,
the VH comprises the amino acid sequence of SEQ ID NO: 8 and the VL comprises the amino acid sequence of SEQ ID NO: 14,
the VH comprises the amino acid sequence of SEQ ID NO: 9 and the VL comprises the amino acid sequence of SEQ ID NO: 14,
the VH comprises the amino acid sequence of SEQ ID NO: 10 and the VL comprises the amino acid sequence of SEQ ID NO: 15,
the VH comprises the amino acid sequence of SEQ ID NO: 11 and the VL comprises the amino acid sequence of SEQ ID NO: 15; or
the VH comprises the amino acid sequence of SEQ ID NO: 12 and the VL comprises the amino acid sequence of SEQ ID NO: 16.

The antibodies or antigen-binding fragments thereof can be of any type (e.g., IgG, IgE, IgM, IgD, IgA, and IgY), class (e.g., IgG1, IgG2, IgG3, IgG4, IgA1, and IgA2), or subclass.

In some embodiments, antibody or antigen-binding fragment thereof is an IgG1 antibody, IgG2 antibody, IgG3 antibody, or IgG4 antibody or antigen-binding fragment thereof.

In some embodiments, the antibody or antigen-binding fragment thereof is an IgG antibody or antigen-binding fragment thereof.

In some embodiments, the antigen-binding fragment include, e.g., Fab, Fab′, F(ab′)₂, Fv fragments and scFv fragments.

In some embodiments, the antibody or antigen-binding fragment thereof is a humanized antibody or antigen-binding fragment thereof.

In some embodiments, the antibody or antigen-binding fragment thereof is a chimeric antibody or antigen-binding fragment thereof.

In non-limiting examples, the humanized antibody is a human antibody in which hypervariable region (e.g., CDR) residues are replaced by hypervariable region (e.g., CDR) residues from a non-human antibody (e.g., a mouse, rat, or rabbit antibody). In some embodiments, the humanized antibody is a human antibody in which the framework region residues are replaced by corresponding non-human (e.g., mouse) framework region residues.

In some embodiments, the humanized antibody can also contain at least a portion of a constant region (Fc) of a human immunoglobulin. Humanized antibodies can be produced using molecular biology methods known in the art.

In some embodiments, the antibody or antigen-binding fragment thereof is polyclonal, monoclonal or multi-specific antibody or antigen-binding fragment thereof.

In some embodiments, the antibody or antigen-binding fragment thereof provided by the disclosure specifically binds to LILRB2, exemplary LILRB2 includes human LILRB2.

In some embodiments, the anti-LILRB2 antibody or antigen-binding fragment thereof can block the signaling pathway and upregulates the immune response. In some embodiments, the antibodies or antigen-binding fragments thereof described herein are LILRB2 agonist. In some embodiments, the antibodies or antigen-binding fragments thereof described herein are LILRB2 antagonist.

In some embodiments, the antibody or antigen-binding fragment thereof specifically binds to LILRB2 with an EC₅₀ (concentration for 50% of maximal effect) value of less than 20 nM, less than 10 nM, less than 5 nM or less than 1 nM measured by the standard techniques in the art, for example, Octet rule, Biacore or FACS.

In some embodiments, the antibody or antigen-binding fragment thereof binds human LILRB2 with KD (affinity constant) of less than 15 nM, less than 10 nM, less than 8 nM, or less than 6 nM measured by the standard techniques in the art, for example, Octet rule, Biacore or FACS.

In general, the KD value, or affinity constant, is the equilibrium dissociation constant, or a ratio of Kd/Ka, between the antibody and its antigen. Kd refers to dissociation constant, and Ka refers to association constant.

Furthermore, the antibody or antigen-binding fragment thereof provided herein blocks the interaction between human LILRB2 and classical MHC (major histocompatibility complex) class I molecules. In some embodiments, the antibody or antigen-binding fragment thereof blocks the interaction between human LILRB2 and HLA-A2. In some embodiments, the antibody or antigen-binding fragment thereof blocks the interaction between human LILRB2 and HLA-G.

In some embodiments, the antibody or antigen-binding fragment thereof blocks the interaction between human LILRB2 and HLA-G with an IC₅₀ value of less than 10 nM, less than 5 nM, less than 1 nM, less than 0.5 nM, or less than 0.3 nM measured by the standard techniques in the art, for example, FACS (Fluorescence Activated Cell Sorting) assay.

In general, the IC₅₀ value represents the concentration at which a substance exerts half of its maximal inhibitory effect. This value is typically used to characterize an antagonist of a biological process.

In some embodiments, the antibody or antigen-binding fragment thereof blocks the interaction between human LILRB2 and HLA-A2 with an IC₅₀ value of less than 20 nM, less than 10 nM, less than 5 nM, less than 1 nM, or less than 0.6 nM measured by the standard techniques in the art, for example, FACS (Fluorescence Activated Cell Sorting) assay.

The antibody or antigen-binding fragment thereof blocks the interaction between LILRB2 (e.g., human LILRB2) and ANGPTL (Angiopoietin-like protein) Families molecules (e.g., human ANGPTL).

In some embodiments, the antibody or antigen-binding fragment thereof blocks the interaction between human LILRB2 and human ANGPTL1 (Angiopoietin-like protein 1).

In some embodiments, the antibody or antigen-binding fragment thereof blocks the interaction between human LILRB2 and human ANGPTL2 (Angiopoietin-like protein 2).

In some embodiments, the antibody or antigen-binding fragment thereof blocks the interaction between human LILRB2 and human ANGPTL3 (Angiopoietin-like protein 3).

In some embodiments, the antibody or antigen-binding fragment thereof blocks the interaction between human LILRB2 and human ANGPTL4 (Angiopoietin-like protein 4).

In some embodiments, the antibody or antigen-binding fragment thereof blocks the interaction between human LILRB2 and human ANGPTL6 (Angiopoietin-like protein 6).

In some embodiments, the antibody or antigen-binding fragment thereof blocks the interaction between human LILRB2 and human ANGPTL7 (Angiopoietin-like protein 7).

In some embodiments, the antibody or antigen-binding fragment thereof blocks the interaction between human LILRB2 and human MAG (myelin associated glycoprotein).

In some embodiments, the antibody or antigen-binding fragment thereof provided herein could not or almost not or probably not bind to LILRA1 (Leukocyte Immunoglobulin Like Receptor A1).

In some embodiments, the antibody or antigen-binding fragment thereof provided herein could not or almost not or probably not bind to LILRA2 (Leukocyte Immunoglobulin Like Receptor A2).

In some embodiments, the antibody or antigen-binding fragment thereof provided herein could not or almost not or probably not bind to LILRA3 (Leukocyte Immunoglobulin Like Receptor A3).

In some embodiments, the antibody or antigen-binding fragment thereof provided herein could not or almost not or probably not bind to LILRA4 (Leukocyte Immunoglobulin Like Receptor A4).

In some embodiments, the antibody or antigen-binding fragment thereof provided herein could not or almost not or probably not bind to LILRA5 (Leukocyte Immunoglobulin Like Receptor A5),

In some embodiments, the antibody or antigen-binding fragment thereof provided herein could not or almost not or probably not bind to LILRB1 (Leukocyte Immunoglobulin Like Receptor B1),

In some embodiments, the antibody or antigen-binding fragment thereof provided herein could not or almost not or probably not bind to LILRB3 (Leukocyte Immunoglobulin Like Receptor B3),

In some embodiments, the antibody or antigen-binding fragment thereof provided herein could not or almost not or probably not bind to LILRB4 (Leukocyte Immunoglobulin Like Receptor B4).

In some embodiments, the antibody or antigen-binding fragment thereof provided herein could not or almost not or probably not bind to LILRB5 (Leukocyte Immunoglobulin Like Receptor B5).

In some embodiments, the antibody or antigen-binding fragment thereof binding to LILRB2 is capable to upregulate the release or enhance the expression of pro-inflammatory cytokines and/or chemokines, without limitation, e.g., IL-2, IFN-γ and/or TNF-α.

In some embodiments, the pro-inflammatory cytokines and/or chemokines are selected from the group consisting of IL-2, IFN-γ, TNF-α, Granzyme B, PDGF-AA, IL-3, IL-1beta, IL-5, TGF-alpha, IFN-alpha, CCL4, FGF-basic, IL-13, G-CSF, IL-4, PD-L1, CCL20, IL-15, GM-CSF, CCL5, CCL3, PDGF-AB, CCL19, CXCL2, IL-6, VEGF, and the combination thereof.

In some embodiments, the antibody or antigen-binding fragment thereof provided herein upregulates the immune response by reducing the expression of anti-inflammatory interleukin, e.g., IL-10.

In some embodiments, the antibody or antigen-binding fragment thereof provided herein is capable of promoting human PBMCs into a pro-inflammation status. In some embodiments, the status of human PBMCs can be measured by detecting the release of anti-inflammatory interleukin, e.g., IL-10, and/or pro-inflammatory cytokines, e.g., TNF-α.

In some embodiments, the antibody or antigen-binding fragment thereof provided herein is capable of promoting human monocytes into a pro-inflammation status. In some embodiments, the status of human PBMCs can be measured by detecting the release of anti-inflammatory interleukin, e.g., IL-10, and/or pro-inflammatory cytokines, e.g., TNF-α.

In some embodiments, the antibody or antigen-binding fragment thereof provided herein is capable of reprogramming human monocyte derived M2 macrophages into pro-inflammation M1 phenotype. In some embodiments, the effects of the above-described antibody or antigen-binding fragment thereof on macrophage phenotype switch were determined by quantitating the percentage of CD86^{high}CD163^{low} population (M1%) and the release of TNFα. CD86 and CD163 are known marker expressed in M1 and M2 macrophages, it's well-known to those skilled in the art to distinguish M1 and M2 macrophages by detecting the expression level of CD86 and CD163.

In some embodiments, the antibody or antigen-binding fragment thereof provided herein is capable of relieving the suppressive function of M2 macrophages. In some embodiments, the effect of relieving the suppressive function of M2 macrophages is measured by detecting the release of IL-2, IFNγ, Granzyme B, TNFα, PDGF-AA, IL-3, IL-1beta, IL-1F2, IL-5, TGF-alpha, IFN-alpha, CCL4, MIP-1 beta, FGF-basic, IL-13, G-CSF, IL-4, B7-H1, PD-L1, CCL20, MIP-3 alpha, IL-15, GM-CSF, CCL5, RANTES, CCL3, MIP-1 alpha, PDGF-AB, PDGF-BB, CCL19, MIP-3 beta, CXCL2, GRO beta, IL-6, and/or VEGF. In some embodiments, the effect of relieving the suppressive function of M2 macrophages is measured by detecting the release of IL-2, IFNγ and TNFα.

In some embodiments, the antibody or antigen-binding fragment thereof provided herein is capable of promoting immature dendritic cells (DCs) into a maturation status. Effects of the above-described antibody or antigen-binding fragment thereof on dendritic cells maturation were determined by quantitating the percentage of CD86^{high}CD163^{low} population (M1%), and the release of pro-inflammation cytokines and chemokines which are selected from the group of TNFα, IL-6, IL-8, CXCL8, IFN-beta, IL-10, CCL2, JE, MCP-1, VEGF, CCL20, MIP-3 alpha, CCL3, MIP-1 alpha, CCL22, MDC, IL-1 alpha, IL-1F1, CCL23, MPIF-1, and the combination thereof.

In some embodiments, the antibody or antigen-binding fragment thereof upregulates the immune response to a LILRB2-related disease, disorder, or condition, e.g., tumor.

In some embodiments, the antibody or antigen-binding fragment thereof upregulates the immune response by reprogramming human monocyte derived M2 macrophages into M1 phenotype. In some embodiments, the antibody or antigen-binding fragment thereof upregulates the immune response by reprogramming tumor-associated macrophages from M2 phenotype to M1 phenotype.

In some embodiments, the antibody or antigen-binding fragment thereof provided herein relieves the suppressive function of M2 macrophages. In some embodiments, the antibody or antigen-binding fragment thereof upregulates the immune response by relieving tumor-associated macrophages M2 phenotype mediated suppression of T cells.

In some embodiments, the antibody or antigen-binding fragment thereof upregulates the immune response by promoting dendritic cells (e.g., human dendritic cells) into a maturation status.

In some embodiments, the antibody or antigen-binding fragment thereof upregulates the immune response by promoting monocytes (e.g., human monocytes) into a pro-inflammation status.

In some embodiments, the antibody or antigen-binding fragment thereof upregulates the immune response by promoting PBMCs (e.g., human PBMCs) into a pro-inflammation status.

In some embodiments, the antibody or antigen-binding fragment thereof blocks LILRB2 binding to tumor-associated macrophages. In some embodiments, LILRB2 blockade reprograms tumor-associated macrophages (TAMs) into a proinflammatory phenotype. In some embodiments, LILRB2 blockade suppresses regulatory T cell (Treg) infiltration, e.g., in tumor microenvironment.

In one aspect, the disclosure also provides a pharmaceutical composition including the antibody or antigen-binding fragment thereof that binds to LILRB, and an anti-PD-1 antibody. Exemplary anti-PD-1 antibody is Keytruda.

The antibody or antigen-binding fragment thereof described above can induced greater release of pro-inflammation cytokines/chemokines in combination with an anti-PD-1 antibody.

The antibody or antigen-binding fragment thereof described above can relieved the suppressive function of M2 macrophages in combination with an anti-PD-1 antibody.

The antibody or antigen-binding fragment thereof described above synergizes with an anti-PD-1 antibody to reinvigorate T cell functionalities.

The antibody or antigen-binding fragment thereof described above can improve the immune response in combination with an anti-PD-1 antibody.

In one aspect, provided herein is an antibody or antigen-binding fragment thereof which binds to the peptide including amino acid sequence set forth in SEQ ID NO: 21. In some embodiments, the peptide of SEQ ID NO: 21 can be the binding epitope on LILRB2. In some embodiments, the peptide of SEQ ID NO: 21 can be partly the binding epitope on LILRB2.

In one aspect, provided herein is a binding epitope on LILRB2, wherein the binding epitope comprises the amino acid sequence set forth in SEQ ID NO: 21. In some embodiments, the LILRB2 is human LILRB2.

In the disclosure, the term of “binding epitope” refers to a molecular region on the surface of an antigen (or therapeutic target) capable of eliciting an immune response and of combining with the specific antibody produced by such a response.

### Polynucleotides, Vectors and Host Cells

The present disclosure provides an isolated polynucleotide encoding the antibody or antigen-binding fragment thereof that binds to LILRB2 or the anti-LILRB2 antibody or antigen-binding fragment thereof.

The polynucleotide is polymers of DNA, RNA, DNA/RNA hybrids, or modifications thereof. In some embodiments, the polynucleotide is polymers of DNA. In some embodiments, the polynucleotide is polymers of RNA.

DNA or RNA encoding the antibody or antigen-binding fragment thereof that binds to LILRB2 is readily isolated and sequenced using conventional procedures (e.g., by using oligonucleotide probes that are capable of binding specifically to genes encoding the heavy and light chains of the antibody). The encoding DNA or RNA may also be obtained by synthetic methods.

The isolated polynucleotide encoding the antibodies or antigen-binding fragments thereof that bind to LILRB2 can be inserted into a vector for further cloning (amplification of the DNA) or for expression, using recombinant techniques known in the art. Many vectors are available. The vector components generally include, but are not limited to, one or more of the following: a signal sequence, an origin of replication, one or more marker genes, an enhancer element, a promoter (e.g., SV40, CMV, EF-1a), and a transcription termination sequence.

In one aspect, provided herein is an isolated vector comprising the polynucleotide encoding the antibody or antigen-binding fragment thereof that binds to LILRB2 or the anti-LILRB2 antibody or antigen-binding fragment thereof.

The present disclosure provides vectors comprising the isolated polynucleotides provided herein. In some embodiments, the vector provided herein encodes the antibodies or antigen-binding fragments thereof, at least one promoter (e.g., SV40, CMV, EF-1a) operably linked to the nucleic acid sequence, and at least one selection marker. Examples of vectors include, but are not limited to, retrovirus (including lentivirus), adenovirus, adeno-associated virus, herpesvirus (e.g., herpes simplex virus), poxvirus, baculovirus, papillomavirus, papovavirus (e.g. SV40), lambda phage, and M13 phage, plasmid pcDNA3.3, pMD18-T, pOptivec, pCMV, pEGFP, pIRES, pQD-Hyg-GSeu, pALTER, pBAD, pcDNA, pCal, pL, pET, pGEMEX, pGEX, pCI, pEGFT, pSV2, pFUSE, pVITRO, pVIVO, pMAL, pMONO, pSELECT, pUNO, pDUO, Psg5L, pBABE, pWPXL, pBI, p15TV-L, pPro18, pTD, pRS10, pLexA, pACT2.2, pCMV-SCRIPT.®, pCDM8, pCDNA1.1/amp, pcDNA3.1, pRc/RSV, PCR 2.1, pEF-1, pFB, pSG5, pXT1, pCDEF3, pSVSPORT, pEF-Bos etc.

Vectors comprising the polynucleotide sequence encoding the antibody or antigen-binding fragment thereof described above can be introduced to a host cell for cloning or gene expression. Suitable host cells for cloning or expressing the DNA or RNA in the vectors herein are the prokaryote, yeast, or higher eukaryote cells described above. Suitable prokaryotes for this purpose include eubacteria, such as Gram-negative or Gram-positive organisms, for example, Enterobacteriaceae such as Escherichia, e.g., E. coli, Enterobacter, Erwinia, Klebsiella, Proteus, Salmonella, e.g., Salmonella typhimurium, Serratia, e.g., Serratia marcescens, and Shigella, as well as Bacilli such as B. subtilis and B. licheniformis, Pseudomonas such as P. aeruginosa, and Streptomyces.

In addition to prokaryotes, eukaryotic microbes such as filamentous fungi or yeast are suitable cloning or expression hosts for anti-LILRB2 antibody-encoding vectors. Saccharomyces cerevisiae, or common baker's yeast, is the most commonly used among lower eukaryotic host microorganisms. However, a number of other genera, species, and strains are commonly available and useful herein, such as Schizosaccharomyces pombe; Kluyveromyces hosts such as, e.g. K. lactis, K. fragilis (ATCC 12, 424), K. bulgaricus (ATCC 16, 045), K. wickeramii (ATCC 24, 178), K. waltii (ATCC 56, 500), K. drosophilarum (ATCC 36, 906), K. thermotolerans, and K. marxianus; yarrowia (EP 402, 226); Pichia pastoris (EP 183, 070); Candida, Trichoderma reesia (EP 244, 234); Neurospora crassa; Schwanniomyces such as Schwanniomyces occidentalis; and filamentous fungi such as, e.g. Neurospora, Penicillium, Tolypocladium, and Aspergillus hosts such as A. nidulans and A. niger.

In one aspect, provided herein is a host cell comprising the isolated polynucleotide described above or the isolated vector described above.

Suitable host cells for the expression of antibodies (e.g., glycosylated antibodies) or antigen-fragment thereof provided herein are derived from multicellular organisms. However, interest has been greatest in vertebrate cells, and propagation of vertebrate cells in culture (tissue culture) has become a routine procedure. Examples of useful mammalian host cell lines are monkey kidney CV1 line transformed by SV40 (COS-7, ATCC CRL 1651); human embryonic kidney line (293 or 293 cells subcloned for growth in suspension culture, Graham et al., J. Gen Virol. 36:59 (1977)); baby hamster kidney cells (BHK, ATCC CCL 10); Chinese hamster ovary cells/-DHFR (CHO, Urlaub et al., Proc. Natl. Acad. Sci. USA 77:4216 (1980)); mouse sertoli cells (TM4, Mather, Biol. Reprod. 23:243-251 (1980)); monkey kidney cells (CV1 ATCC CCL 70); African green monkey kidney cells (VERO-76, ATCC CRL-1587); human cervical carcinoma cells (HELA, ATCC CCL 2); canine kidney cells (MDCK, ATCC CCL 34); buffalo rat liver cells (BRL 3A, ATCC CRL 1442); human lung cells (W138, ATCC CCL 75); human liver cells (Hep G2, HB 8065); mouse mammary tumor (MMT 060562, ATCC CCL51); TRI cells (Mather et al., Annals N.Y. Acad. Sci. 383:44-68 (1982)); MRC 5 cells; FS4 cells; and a human hepatoma line (Hep G2). In some embodiments, the host cell is a mammalian cultured cell line, such as CHO, BHK, NS0, 293 and their derivatives.

Host cells are transformed with the above-described expression or cloning vectors for producing antibody or antigen-binding fragment thereof that binds to LILRB2 or the anti-LILRB2 antibody or antigen-binding fragment thereof and cultured in conventional nutrient media modified as appropriate for inducing promoters, selecting transformants, or amplifying the genes encoding the desired sequences. In another embodiment, the antibody may be produced by homologous recombination known in the art. In certain embodiments, the host cell is capable to produce the antibody or antigen-binding fragment thereof provided herein.

In one aspect, the present disclosure also provides a method of expressing the antibody or an antigen-binding fragment thereof provided herein, comprising culturing the host cell provided herein under the condition at which the vector of the present disclosure is expressed. The host cells used to produce the antibodies or antigen-binding fragments thereof provided herein may be cultured in a variety of media. Commercially available media such as Ham's F10 (Sigma), Minimal Essential Medium (MEM), (Sigma), RPMI-1640 (Sigma), and Dulbecco's Modified Eagle's Medium (DMEM), Sigma) are suitable for culturing the host cells. In addition, any of the media described in Ham et al., Meth. Enz. 58:44 (1979), Barnes et al., Anal. Biochem. 102:255 (1980), U.S. Pat. Nos. 4,767,704; 4,657,866; 4,927,762; 4,560,655; or 5,122,469; WO 90/03430; WO 87/00195; or U.S. Pat. Re. 30, 985 may be used as culture media for the host cells. Any of these media may be supplemented as necessary with hormones and/or other growth factors (such as insulin, transferrin, or epidermal growth factor), salts (such as sodium chloride, calcium, magnesium, and phosphate), buffers (such as HEPES), nucleotides (such as adenosine and thymidine), antibiotics (such as GENTAMYCIN™ drug), trace elements (defined as inorganic compounds usually present at final concentrations in the micromolar range), and glucose or an equivalent energy source. Any other necessary supplements may also be included at appropriate concentrations that would be known to a person skilled in the art. The culture conditions, such as temperature, pH, and the like, are those previously used with the host cell selected for expression, and will be apparent to a person skilled in the art.

### Pharmaceutical Compositions

In one aspect, the present disclosure further provides pharmaceutical compositions comprising the antibody or antigen-binding fragment thereof described above, and one or more pharmaceutically acceptable carriers. In some embodiments, the present disclosure provides a pharmaceutical composition containing the antibody or antigen-binding fragment thereof described above, and an anti-PD-1 antibody (e.g., Keytruda).

In some embodiments, the present disclosure further provides pharmaceutical compositions comprising an isolated polynucleotide encoding the antibody or antigen-binding fragment thereof that binds to LILRB2, and one or more pharmaceutically acceptable carriers.

In some embodiments, the present disclosure further provides pharmaceutical compositions comprising an isolated vector comprising the polynucleotide encoding the antibody or antigen-binding fragment thereof that binds to LILRB2 or the anti-LILRB2 antibody or antigen-binding fragment thereof, and one or more pharmaceutically acceptable carriers.

In some embodiments, the present disclosure further provides pharmaceutical compositions comprising a host cell, which contains the polynucleotide encoding the antibody or antigen-binding fragment thereof that binds to LILRB2 (anti-LILRB2 antibody or antigen-binding fragment thereof) or the vector comprising the polynucleotide described above.
pharmaceutical compositions provided herein may be formulated in any manner known in the art, such as, pharmaceutical compositions provided herein can be formulated for parenteral (e.g., intravenous, intraarterial, intramuscular, intradermal, subcutaneous, or intraperitoneal) administration in dosage unit form (i.e., physically discrete units containing a predetermined quantity of active compound for ease of administration and uniformity of dosage).

Pharmaceutical compositions are formulated to be compatible with their intended route of administration (e.g., intravenous, intraarterial, intramuscular, intradermal, subcutaneous, or intraperitoneal).

Pharmaceutical acceptable carriers for use in the pharmaceutical compositions disclosed herein may include, for example, pharmaceutically acceptable liquid, gel, or solid carriers, aqueous vehicles, nonaqueous vehicles, antimicrobial agents, isotonic agents, buffers, antioxidants, anesthetics, suspending/dispending agents, sequestering or chelating agents, diluents, adjuvants, excipients, or non-toxic auxiliary substances, other components known in the art, or various combinations thereof.

Suitable components may include, for example, antioxidants, fillers, binders, disintegrants, buffers, preservatives, lubricants, flavorings, thickeners, coloring agents, emulsifiers or stabilizers such as sugars and cyclodextrins. Suitable antioxidants may include, for example, methionine, ascorbic acid, EDTA, sodium thiosulfate, platinum, catalase, citric acid, cysteine, thioglycerol, thioglycolic acid, thiosorbitol, butylated hydroxyanisole, butylated hydroxytoluene, and/or propyl gallate. As disclosed herein, inclusion of one or more antioxidants such as methionine in a composition comprising an antibody or antigen-binding fragment thereof and conjugates provided herein decreases oxidation of the antibody or antigen-binding fragment thereof. This reduction in oxidation prevents or reduces loss of binding affinity, thereby improving antibody stability and maximizing shelf-life. Therefore, in certain embodiments, pharmaceutical compositions are provided that comprise one or more antibodies or antigen-binding fragments thereof as disclosed herein and one or more antioxidants such as methionine.

To further illustrate, pharmaceutical acceptable carriers may include, for example, aqueous vehicles such as sodium chloride injection, Ringer's injection, isotonic dextrose injection, sterile water injection, or dextrose and lactated Ringer's injection, nonaqueous vehicles such as fixed oils of vegetable origin, cottonseed oil, corn oil, sesame oil, or peanut oil, antimicrobial agents at bacteriostatic or fungistatic concentrations, isotonic agents such as sodium chloride or dextrose, buffers such as phosphate or citrate buffers, antioxidants such as sodium bisulfate, local anesthetics such as procaine hydrochloride, suspending and dispersing agents such as sodium carboxymethylcellulose, hydroxypropyl methylcellulose, or polyvinylpyrrolidone, emulsifying agents such as Polysorbate 80 (Tween-80), sequestering or chelating agents such as EDTA (ethylenediaminetetraacetic acid) or EGTA (ethylene glycol tetra acetic acid), ethyl alcohol, polyethylene glycol, propylene glycol, sodium hydroxide, hydrochloric acid, citric acid, or lactic acid. Antimicrobial agents utilized as carriers may be added to pharmaceutical compositions in multiple-dose containers that include phenols or cresols, benzyl alcohol, chlorobutanol, methyl and propyl p-hydroxybenzoic acid esters, thimerosal, benzalkonium chloride and benzethonium chloride. Suitable excipients may include, for example, water, saline, dextrose, glycerol, or ethanol. Suitable non-toxic auxiliary substances may include, for example, wetting or emulsifying agents, pH buffering agents, stabilizers, solubility enhancers, or agents such as sodium acetate, sorbitan monolaurate, triethanolamine oleate, or cyclodextrin.

In some embodiments, the pharmaceutical compositions can be a liquid solution, suspension, or emulsion.

In some embodiments, the pharmaceutical compositions are formulated into an injectable composition. The injectable pharmaceutical compositions may be prepared in any conventional form, such as for example liquid solution, suspension, emulsion, or solid forms suitable for generating liquid solution, suspension, or emulsion. Preparations for injection may include sterile and/or non-pyretic solutions ready for injection, sterile dry soluble products, such as lyophilized powders, ready to be combined with a solvent just prior to use, including hypodermic tablets, sterile suspensions ready for injection, sterile dry insoluble products ready to be combined with a vehicle just prior to use, and sterile and/or non-pyretic emulsions. The solutions may be either aqueous or nonaqueous.

In some embodiments, unit-dose parenteral preparations are packaged in an ampoule, a vial or a syringe with a needle. All preparations for parenteral administration should be sterile and not pyretic, as is known and practiced in the art.

In some embodiments, a sterile, lyophilized powder is prepared by dissolving an antibody or antigen-binding fragment as disclosed herein in a suitable solvent. The solvent may contain an excipient which improves the stability or other pharmacological components of the powder or reconstituted solution, prepared from the powder. Excipients that may be used include, but are not limited to, water, dextrose, sorbitol, fructose, corn syrup, xylitol, glycerin, glucose, sucrose or other suitable agents. The solvent may contain a buffer, such as citrate, sodium or potassium phosphate or other such buffer known to a person skilled in the art at, in one embodiment, about neutral pH. Subsequent sterile filtration of the solution followed by lyophilization under standard conditions known to a person skilled in the art provides a desirable formulation. In one embodiment, the resulting solution will be apportioned into vials for lyophilization. Each vial can contain a single dosage or multiple dosages of the antibody or antigen-binding fragment thereof that binds to LILRB2 or composition thereof. Overfilling vials with a small amount above that needed for a dose or set of doses (e.g., about 5%, 10%, or 15%) is acceptable to facilitate accurate sample withdrawal and accurate dosing. The lyophilized powder can be stored under appropriate conditions, such as at about 4° C. to room temperature.

Reconstitution of a lyophilized powder with water for injection provides a formulation for use in parenteral administration. In one embodiment, for reconstitution the sterile and/or non-pyretic water or other liquid suitable carrier is added to lyophilized powder. The precise amount depends upon the selected therapy being given and can be empirically determined.

### Kits

In one aspect, the present disclosure provides a kit comprising the antibody or an antigen-binding fragment thereof described above, the isolated polynucleotide described above, the isolated vector described above, and/or the pharmaceutical composition provided herein.

In some embodiments, the present disclosure provides a kit comprising the antibody or an antigen-binding fragment thereof provided herein, and a second therapeutic agent. In some embodiments, the second therapeutic agent is selected from the group consisting of a chemotherapeutic agent, an anti-cancer drug, radiation therapy, an immunotherapy agent, an anti-angiogenesis agent, a targeted therapy, a cellular therapy, a gene therapy, a hormonal therapy, an antiviral agent, an antibiotic, an analgesic, an antioxidant, a metal chelator, and cytokines.

Such kits can further include, if desired, one or more of various conventional pharmaceutical kit components, such as, for example, containers with one or more pharmaceutically acceptable carriers, additional containers etc., as will be readily apparent to a person skilled in the art. Instructions, either as inserts or a labels, indicating quantities of the components to be administered, guidelines for administration, and/or guidelines for mixing the components, can also be included in the kit.

In some embodiments, the present disclosure provides kits comprising the antibody or antigen-binding fragment thereof provided herein and/or the pharmaceutical composition provided herein, optionally conjugated with a detectable moiety, which is useful in detecting a LILRB2 related disease, disorder or condition. The kits may further comprise instructions for use.

### Use in Manufacture of Therapeutic Agent

The disclosure provides the use of the antibody or antigen-binding fragment thereof, the isolated polynucleotide, the isolated vector, the pharmaceutical compositions, or the kit described above in the manufacture of a therapeutic agent for binding to human LILRB2.

In some embodiments, provided herein is the use of the antibody or antigen-binding fragment thereof in the manufacture of a drug for blocks the interaction between human LILRB2 and HLA-A2, HLA-G, ANGPTL1, ANGPTL2, ANGPTL3, ANGPTL4, ANGPTL6, ANGPTL7, and/or MAG.

In some embodiments, the therapeutic agents include, without limitation, drugs, antibodies, fusion proteins, vaccines, CAR-T and antibody-drug conjugate. In some embodiments, the therapeutic agent is a biologically active substance, which can treat or relieve a LILRB2-related disease, disorder, or condition.

In some embodiments, provided herein is the use of the antibody or antigen-binding fragment thereof, the isolated polynucleotide, the isolated vector, the pharmaceutical compositions, or the kit in the manufacture of a therapeutic agent for upregulating or enhancing the immune response to a disease, disorder, or condition, e.g., tumor.

In some embodiments, provided herein is the use of the antibody or antigen-binding fragment thereof, the isolated polynucleotide, the isolated vector, the pharmaceutical compositions, or the kit in the manufacture of a therapeutic agent for upregulating or enhancing the immune response to a LILRB2-related disease, disorder, or condition, e.g., tumor.

In some embodiments, provided herein is the use of the antibody or antigen-binding fragment thereof, the isolated polynucleotide, the isolated vector, the pharmaceutical compositions, or the kit described above in the manufacture of a therapeutic agent for enhancing immune response to a tumor.

In some embodiments, provided herein is the use of the antibody or antigen-binding fragment thereof described above in the manufacture of a therapeutic agent for enhancing immune response by reprogramming human monocyte derived M2 macrophages into M1 phenotype.

In some embodiments, provided herein is the use of the antibody or antigen-binding fragment thereof described above in the manufacture of a therapeutic agent for relieving the suppressive function of M2 macrophages.

In some embodiments, provided herein is the use of the antibody or antigen-binding fragment thereof described above in the manufacture of a therapeutic agent for enhancing immune response by reprogramming tumor-associated macrophages from M2 phenotype into M1 phenotype macrophages, relieving tumor-associated macrophages M2 phenotype mediated suppression of T cells.

In some embodiments, provided herein is the use of the antibody or antigen-binding fragment thereof described above in the manufacture of a therapeutic agent for promoting PBMCs into a pro-inflammation status. Furthermore, in some embodiments, provided herein is the use of the antibody or antigen-binding fragment thereof described above in the manufacture of a therapeutic agent for promoting human PBMCs into a pro-inflammation status.

In some embodiments, provided herein is the use of the antibody or antigen-binding fragment thereof described above in the manufacture of a therapeutic agent for promoting dendritic cells (e.g., human dendritic cells) into a maturation status.

In some embodiments, provided herein is the use of the antibody or antigen-binding fragment thereof described above in the manufacture of a therapeutic agent for promoting monocytes (e.g., human monocytes) into a pro-inflammation status.

In some embodiments, provided herein is the use of the antibody or antigen-binding fragment thereof described above in the manufacture of a therapeutic agent for promoting PBMCs (e.g., human PBMCs) into a pro-inflammation status.

In some embodiments, the antibody or antigen-binding fragment thereof provided herein enhances immune response by upregulating the expression of pro-inflammatory cytokines, including but not limited to IL-2, IFN-γ, TNF-α, Granzyme B, PDGF-AA, IL-3, IL-1beta, IL-5, TGF-alpha, IFN-alpha, CCL4, FGF-basic, IL-13, G-CSF, IL-4, PD-L1, CCL20, IL-15, GM-CSF, CCL5, CCL3, PDGF-AB, CCL19, CXCL2, IL-6 and VEGF.

In some embodiments, the antibody or antigen-binding fragment thereof provided herein enhances immune response by downregulating the expression of anti-inflammatory interleukin, such as IL-10.

The disclosure provides the use of the antibody or antigen-binding fragment thereof described above, the isolated polynucleotide described above, the isolated vector described above, or the pharmaceutical compositions described above, or the kit described above in the manufacture of a therapeutic agent for diagnosing, preventing or treating a disease, disorder, or condition.

In some embodiments, the disease, disorder or condition is a LILRB2 related disease.

In some embodiments, the disease comprises a tumor, without limitation, the tumor can be hematologic tumor or solid tumor. In some embodiments, the tumor can be malignant or benign. In some embodiments, the disease is a cancer, such as lymphoma.

### Methods of Treatment

The present disclosure provides methods of diagnosing or preventing or treating a LILRB2 related disease, disorder or condition in a subject, comprising administering to the subject a therapeutically effective amount of the antibody or antigen-binding fragment thereof provided herein, the isolated polynucleotide provided herein, the isolated vector provided herein, the host cell provided herein, the pharmaceutical composition provided herein, and/or the kit provided herein.

In some embodiments, the LILRB2 related disease, disorder or condition is characterized in expressing or over-expressing of LILRB2.

In some embodiments, the LILRB2 related disease, disorder or condition comprises a cancer. In some embodiments, the cancer is a LILRB2 expressing cancer. The term “LILRB2 expressing cancer” refers to a cancer characterized in expressing LILRB2 protein in a cancer cell, a tumor infiltrating immune cell or an immune suppression cell, or expressing LILRB2 in a cancer cell, a tumor infiltrating immune cell or an immune suppression cell at a level significantly higher than that would have been expected of a normal cell.

In some embodiments, the subject has been identified as having a cancer cell or tumor infiltrating immune cells or immune suppression cells expressing LILRB2, optionally at a level significantly higher from the level normally found on non-cancer cells or non-immune suppression cells.

The present disclosure further provides a method of enhancing immune response in a subject in need thereof, comprising administrating to the subject a therapeutically effective amount of the antibody or antigen-binding fragment thereof that binds to LILRB2, or the isolated polynucleotide provided herein, or the isolated vector provided herein, or the host cell provided herein, or the pharmaceutical composition provided herein, or the kit provided herein.

In some embodiments, the antibody or antigen-binding fragment thereof that binds to LILRB2, or the isolated polynucleotide provided herein, or the isolated vector provided herein, or the host cell provided herein, or the pharmaceutical composition provided herein, or the kit provided herein upregulate the expression of pro-inflammatory cytokines and/or downregulate the expression of anti-inflammatory interleukin.

Without limitation, in some embodiments, the expression level of IL-2, IFN-γ and/or TNF-α is enhanced. In some embodiments, the expression level of IL-10 is reduced.

In some embodiments, the subject can benefit from modulation of LILRB2 activity.

In some embodiments, the subject is mammals. In some embodiments, the subject comprises human. In some embodiments, the subject is human (or human patients) and can be adult humans or juvenile (e.g., humans below the age of 18 years old). In some embodiments, the subject is non-human animals, including but not limited to non-human primates (e.g., monkey, chimpanzee, gorilla, and the like), rodents (e.g., rats, mice, rabbits), swine (e.g., pig), equine, canine, feline, bovine, and other domestic, farm, and zoo animals.

The therapeutically effective amount of an antibody or antigen-binding fragment provided herein will depend on various factors known in the art, such as for example body weight, age, past medical history, present medications, state of health of the subject and potential for cross-reaction, allergies, sensitivities and adverse side-effects, as well as the administration route and extent of disease development. Dosages may be proportionally reduced or increased by a person skilled in the art (e.g., physician or veterinarian) as indicated by these and other circumstances or requirements.

In certain embodiments, the antibody or antigen-binding fragment provided herein may be administered at a therapeutically effective dosage of about 0.01 mg/kg to about 100 mg/kg. In certain embodiments, the administration dosage may change over the course of treatment. For example, in certain embodiments the initial administration dosage may be higher than subsequent administration dosages. In certain embodiments, the administration dosage may vary over the course of treatment depending on the reaction of the subject.

Dosage regimens may be adjusted to provide the optimum desired response (e.g., a therapeutic response). For example, a single dose may be administered, or several divided doses may be administered over time.

The antibodies or antigen-binding fragments thereof provided herein may be administered by any route known in the art, such as for example parenteral (e.g., subcutaneous, intraperitoneal, intravenous, including intravenous infusion, intramuscular, or intradermal injection) or non-parenteral (e.g., oral, intranasal, intraocular, sublingual, rectal, or topical) routes.

### EXAMPLES

### Example 1. Reagents Generation

### 1.1 Reference Antibodies

The reference antibodies 1E1 and J-19 have been generated according to patent US 2018/0298096 A1 (SEQ ID NO: 69 and 77) and WO 2019/126514 A2 (SEQ ID NO: 13 and 14), respectively. The variable region sequences of 1E1 and J-19 are shown in Table 1. They are both human IgG4 monoclonal antibodies with the S228P mutation in the constant region.

TABLE 1
Variable region sequences of 1E1 and J-19

| Antibody | VH | VL |
|---|---|---|
| 1E1 | QVQLQQWGAGLLKPS | QSVLTQPPSVSGAPG |
| | ETLSLTCAVYGGSFS | QRVTISCTGSSSNIG |
| | GYYWSWIRQPPGKGL | AGYDVHWYQQLPGTA |
| | EWIGEINHSGSTNYN | PKLLIYGNSNRPSGV |
| | PSLKSRVTISVDTSK | PDRFSVSKSGASASL |
| | NQFSLKLSSVTAADT | AITGLQAEDEADYYC |
| | AVYYCARLPTRWVTT | QSFDNSLSAYVFGGG |
| | RYFDLWGRGTLVTVS | TQLTVL |
| | S | (1E1VL, SEQ ID |
| | (1E1VH, SEQ ID | NO: 18) |
| | NO: 17) | |
| J-19 | QVTLKESGPGILQPS | DIQMTQSPASLSTFL |
| | HTLSLTCSFSGFSLN | GEPVTIECRASEDIY |
| | TYAMGVSWIRQPSGK | NDLAWYQQKPGKSPQ |
| | GLEWLASIWWNGNKY | LLIYNANSLHTGVPS |
| | NNPSLKSRLTVSKDT | RFSGSGSGTQYSLKI |
| | SNNQAFLKVTSVDTA | NSLQSEDVASYFCQQ |
| | DTATYYCAHSRIIRF | YYDYPLTFGSGTKLE |
| | TDYVMDAWGQGASVT | IK |
| | VSS | (J-19VL, SEQ ID |
| | (J-19VH, SEQ ID | NO: 20) |
| | NO: 19) | |

### 1.2 Stable Cell Lines

Human LILRB2 (SEQ ID NO: 23) stably expressing cell lines, 293-F/LILRB2 and EL4/LILRB2, have been generated for hybridoma screening and in vitro assays. For 293-F/LILRB2 generation, HEK293-F cells were transfected with GFP-tagged human LILRB2 expression plasmid (Sino Biological) and selectively cultured in medium containing 200 μg/mL hygromycin for 2 weeks. Single cell clones were then isolated by limiting dilution and screened by FACS to obtain the monoclonal cell lines stably expressing human LILRB2. For EL4/LILRB2 stable pool generation, EL4 cells were transfected with non-tagged human LILRB2 expression plasmid (Genery) and selectively cultured in medium containing 500 μg/mL hygromycin.

### 1.3 Recombinant Proteins

Human LILRB2 extracellular domain (ECD) recombinant proteins with human Fc tag (AAH36827.1, Gly24-His458) or 6×His-tag (Q8N423.4, Gln22-His458) were purchased from R & D systems for immunization and hybridoma screening.

A series of recombinant proteins of LILRA/LILRB family members and potential ligands of LILRB2 have also been purchased for in vitro assays. Human LILRA1 (NP_006854.1, Met1-Asn461), LILRA3 (AAH28208.1, Met1-Glu439), LILRA4 (P59901.2, Met1-Asn446), LILRA5 (NP_067073.1, Met1-Arg268), LILRB1 (ADJ55949.1, Met1-His458), LILRB2 (AAH36827.1, Met1-Val461), LILRB3 (AAI04994.1, Met1-Glu443), LILRB4 (AAI04994.1, Met1-Glu443), LILRB5 (NP_006831.1, Met1-Gly458), and MAG (P20916-1, Met1-Pro516) ECD recombinant proteins with 6×His-tag were purchased from Sino Biological. The recombinant proteins of human LILRA2 ECD (NP_001124389, Gly24-Asn449), ANGPTL2 (Q9UKU9-1, Thr260-His493), ANGPTL4 (Q9BY76, Gly26-Ser406, Lys163Ala, Arg164Ala), ANGPTL7 (043827, Met1-Pro346) with His-tag and ANGPTL1 (095841, Gly24-Asp491) with Flag tag were purchased from R & D systems. The recombinant proteins of HLA-A2 complex (MHC-HM431) and HLA-G complex tetramer (HLG-HM41CT) were purchased from Kactus.

### Example 2. Hybridoma Development and Screening

### 2.1 Immunization and Fusion

Four mice from different strains (CD-1, NZB/w, C57BL/6, SJL) were immunized with Fc-tagged human LILRB2 ECD recombinant protein using a quick immunization strategy. Using 6×His-tagged human LILRB2 ECD recombinant protein as antigen, serum titers of the immunized mice were detected by ELISA assay. Final boost was conducted when the serum titers reached a high level. Three days after the final boost, pooled splenocytes and lymph node cells were harvested and fused with SP2/0 mouse myeloma cells. The fused cells were then seeded into 384-well plates for screening.

### 2.2 Primary and Secondary Screening

10-12 days after fusion, supernatants harvested from each well of hybridoma cells were primarily screened by ELISA assay using 6×His-tagged human LILRB2 ECD recombinant protein as antigen. The hybridoma cells in the positive wells were expanded and a secondary confirmation screening was performed using the same assay as the primary screening. Then the hybridoma cells secreting antibodies with top human LILRB2 binding activity were subcloned.

### 2.3 Hybridoma Subcloning and Screening

The selected hybridoma cells were limited diluted into 96-well plates at the density of 1 cell/well to obtain monoclonal hybridoma cells. Supernatants harvested from these monoclonal cells were screened by the same ELISA assay as in example 2.2, and FACS assay using 293-F/LILRB2.

Antibodies secreted from positive clones were quantified by Bio-Layer Interferometry and then assayed for human LILRB2 binding affinity and the activity to block the interaction between LILRB2 and its ligands (refer to the methods described in example 3.2.4). As shown in Table 2, the monoclonal antibody secreting from clone 8G2F10 showed nanomolar human LILRB2 binding affinity and good blocking activity.

TABLE 2
8G2F10 characterization summary

| Clone | ka | kd | KD | Ligand(s)/LILRB2 interaction blockade (%) | | | |
|---|---|---|---|---|---|---|---|
| ID | (1/Ms) | (1/s) | (nM) | HLA-G | HLA-A2 | ANGPTL1 | ANGPTL2 |
| 8G2F10 | 1.04E+06 | 1.39E−03 | 1.35 | 100 | 99 | 100 | 100 |

Heavy chain variable region (VH) and light chain variable region (VL) of clone 8G2F10 secreted monoclonal antibody was sequenced. Their sequences are shown as SEQ ID NO: 12 (VH) and SEQ ID NO: 16 (VL).

### Example 3. Chimeric Antibody Generation and Characterization

### 3.1 Chimeric Antibody Generation

According to the sequencing results (VH, SEQ ID NO: 12; VL, SEQ ID NO: 16) of monoclonal antibody secreted by clone 8G2F10, a human IgG4 chimeric antibody with S228P mutation at Fc region was generated and named as 043c, where the suffix “c” stands for chimeric.

### 3.2 Chimeric Antibody Characterization

### 3.2.1 Binding Activity

Human LILRB2 binding activity of 043c and two benchmark antibodies (1E1 and J-19) was detected by ELISA assay using 6×His-tagged human LILRB2 ECD recombinant protein as antigen. Briefly, 0.5 μg/mL anti-LILRB2 antibodies were incubated in human LILRB2 ECD recombinant protein-coated ELISA plate at 37° C. for 1 hour. After washing, horseradish peroxidase (HRP) labeled detection antibody was added and incubated at 37° C. for 1 hour. Color development was conducted by the addition of 100 μl/well of TMB (3,3′,5,5′-Tetramethylbenzidine) solution. After incubation at room temperature (RT) for 10-15 minutes, the reaction was stopped by adding 100 μl 1N HCl. Then the plates were read immediately using plate reader for optical density at 450 nm. As shown in FIG. 1, 043c, 1E1 and J-19 strongly bound to human LILRB2 ECD recombinant protein.

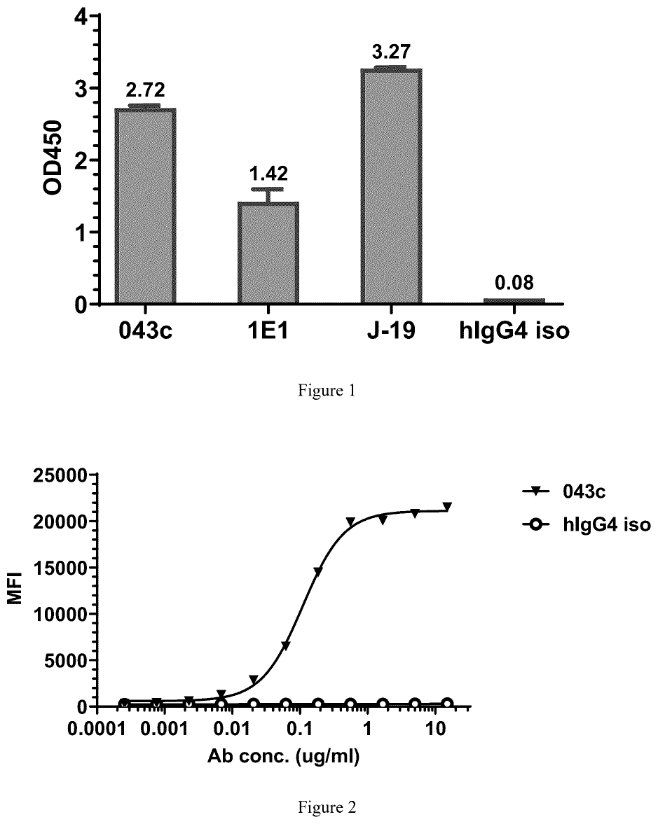

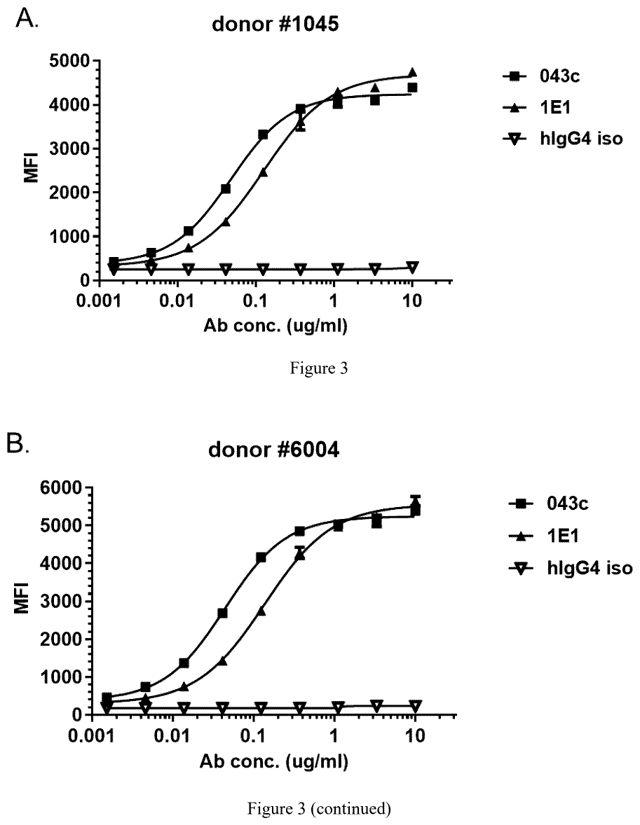

The binding of 043c to cell membrane human LILRB2 was detected by FACS assay using EL4/LILRB2 and human primary monocytes. 043c strongly bound to EL4/LILRB2 cells (FIG. 2) and primary monocytes isolated from healthy human donors (Donor #1045 and #6004, FIG. 3). The EC₅₀ and top MFI values analyzed using Four-Parameter nonlinear fitting by GraphPad Prism 9.0 are summarized in Table 3.

TABLE 3
Binding of 043c and 1E1 to membrane LILRB2

| | | |
|---|---|---|
| EL4/LILRB2 | Donor #1045 | Donor #6004 |

| Ab | EC₅₀ | TOP | EC₅₀ | TOP | EC₅₀ | TOP |
|---|---|---|---|---|---|---|
| code | (nM) | MFI | (nM) | MFI | (nM) | MFI |
| 043c | 0.73 | 21111 | 0.32 | 4240 | 0.29 | 5240 |

| | | | | | |
|---|---|---|---|---|---|
| 1E1 | Not applicable | 0.84 | 4690 | 0.91 | 5558 |

### 3.2.2 Affinity Detection

The binding affinity of 043c, 1E1 and J-19 to human LILRB2 ECD recombinant protein was determined using Bio-Layer Interferometry (Octet). The association and dissociation curves were fitted with 1:1 binding model, and the Ka/Kd/KD values for each antibody were calculated and summarized in Table 4. 043c (KD=0.74 nM) showed a higher antigen binding affinity than 1E1 (KD=8.07 nM) and J-19 (KD=2.14 nM) in the test.

TABLE 4
Human LILRB2 binding affinity of 043c, 1E1 and J-19

| Ab code | ka (1/Ms) | kd (1/s) | KD (nM) |
|---|---|---|---|
| 043c | 7.56E+05 | 5.57E−04 | 0.74 |
| 1E1 | 2.53E+05 | 2.04E−03 | 8.07 |
| J-19 | 8.30E+05 | 1.78E−03 | 2.14 |

### 3.2.3 Binding Specificity

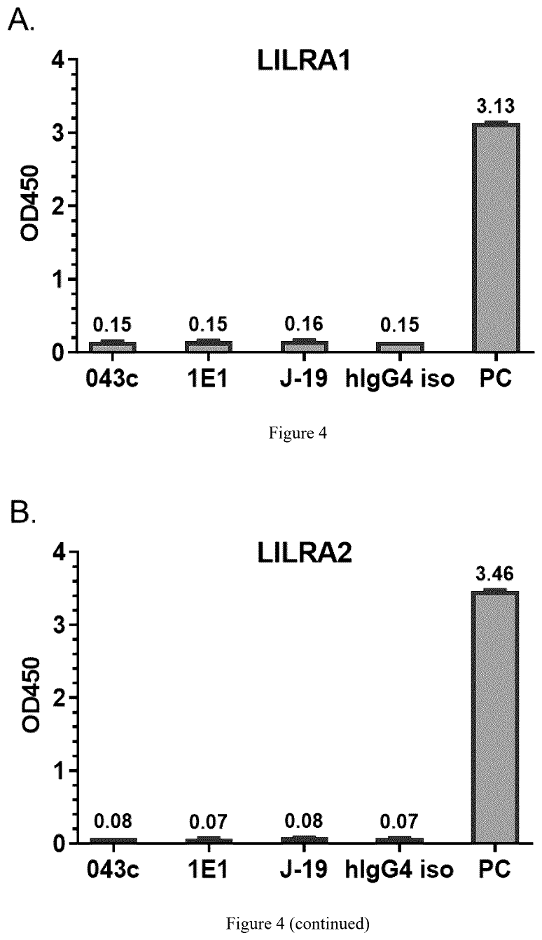

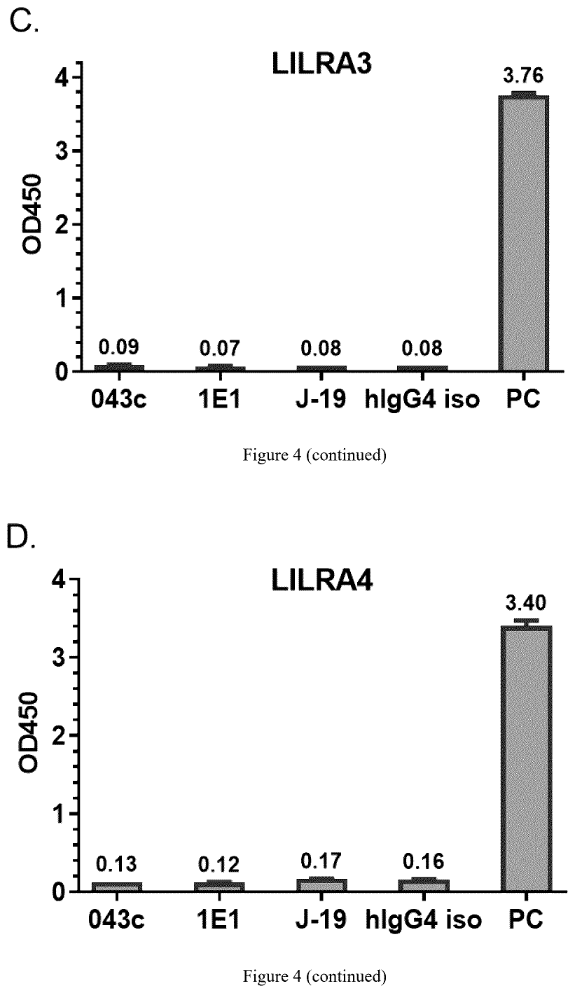

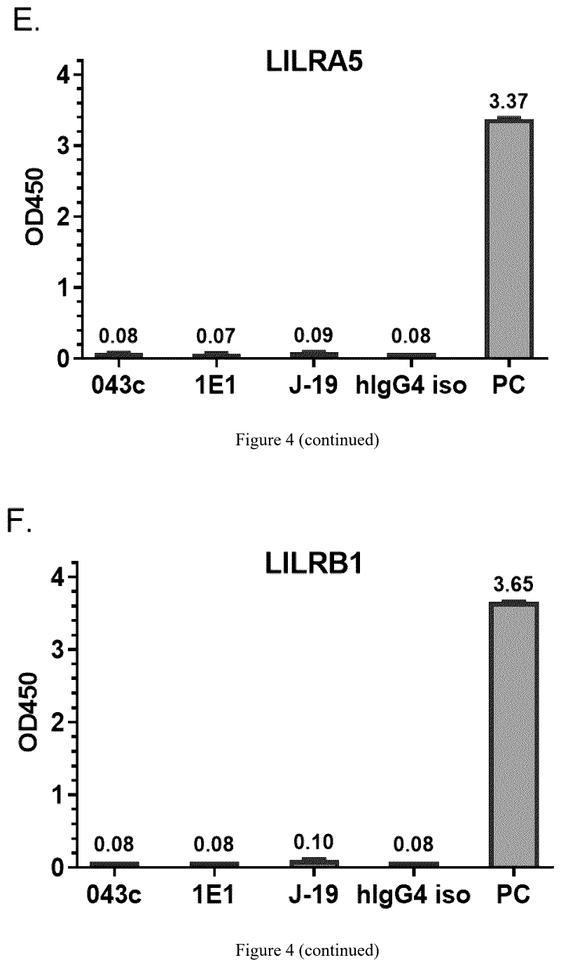

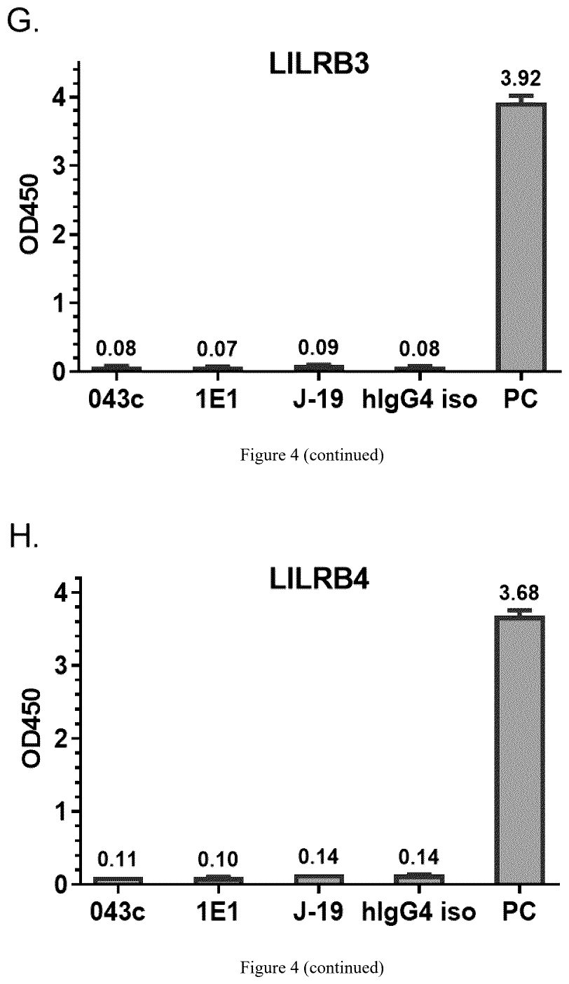

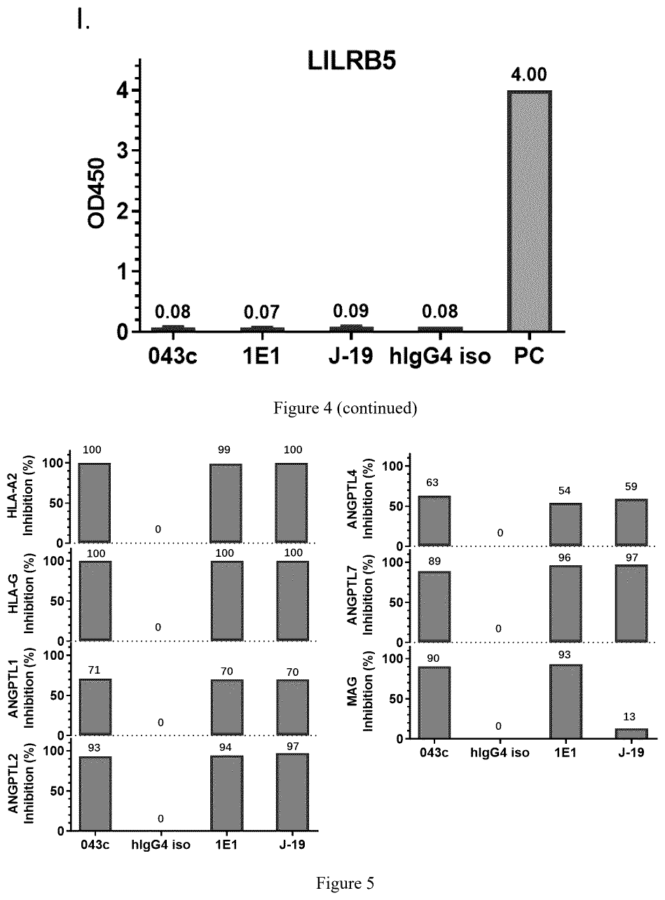

The binding of 043c, 1E1 and J-19 to LILRA/LILRB family members were detected by ELISA assay using LILRA1 (FIG. 4A), LILRA2 (FIG. 4B), LILRA3 (FIG. 4C), LILRA4 (FIG. 4D), LILRA5 (FIG. 4E), LILRB1 (FIG. 4F), LILRB3 (FIG. 4G), LILRB4 (FIG. 4H), LILRB5 (FIG. 4I) ECD recombinant proteins as antigens (refer to method described in example 3.2.1). 043c, 1E1 and J-19 didn't bind to other LILRA/LILRB family members except LILRB2, demonstrating good binding specificity.

### 3.2.4 Blocking Activities

The activities of 043c, 1E1 and J-19 to block the interaction between LILRB2 and its ligands were assessed by competitive FACS. Briefly EL4/LILRB2 cells were preincubated with 10 μg/mL 043c, 1E1, J-19 or IgG4 Isotype for 30 minutes. Then the recombinant proteins of HLA-A2 complex, HLA-G complex tetramer, ANGPTL1, ANGPTL2, ANGPTL4, ANGPTL7, or MAG ECD were added for another 30 minutes incubation. The blocking activities were determined by quantitating the blockade of the ligands binding to EL4/LILRB2 cells. As shown in FIG. 5, 043c, 1E1 and J-19 can almost fully block the interactions between LILRB2 and HLA-A2, HLA-G, ANGPTL2 or ANGPTL7; partially block the interactions between LILRB2 and ANGPTL1 or ANGPTL4. 043c and 1E1 also showed strong LILRB2/MAG interaction blocking activity, while J-19 didn't work well to block this interaction.

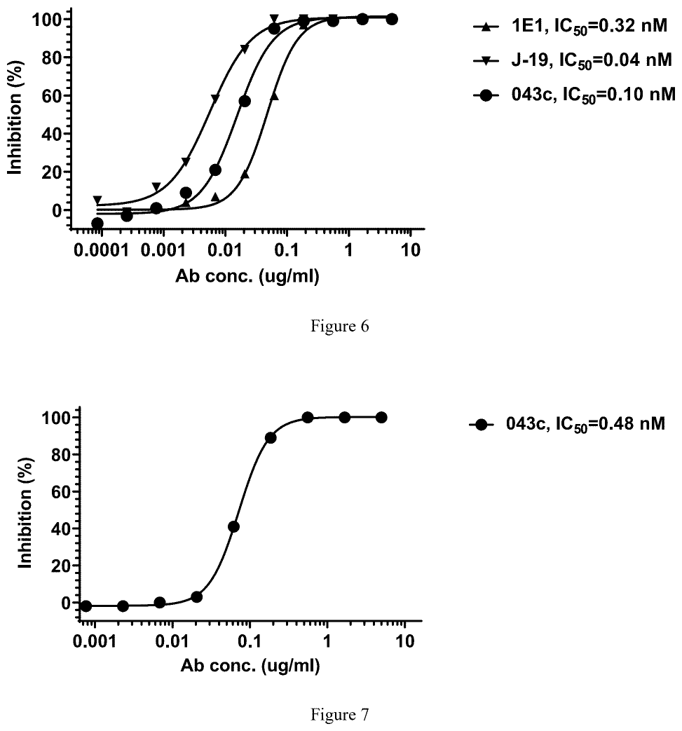

In addition, the titration tests were conducted to further evaluate the activities of 043c, 1E1 and J-19 to block the interactions between LILRB2 and MHC class I ligands (FIG. 6 and FIG. 7). The IC₅₀ values and top blocking ratios analyzed using Four-Parameter nonlinear fitting by GraphPad Prism 9.0 are summarized in Table 5.

TABLE 5
Blocking activities of 043c, 1E1 and J-19

| | LILRB2/HLA-G interaction | | LILRB2/HLA-A2 interaction | |
|---|---|---|---|---|
| Ab | blockade | | blockade | |
| code | IC₅₀ (nM) | TOP Blocking (%) | IC₅₀ (nM) | TOP Blocking (%) |
| 043c | 0.10 | 100 | 0.48 | 100 |
| 1E1 | 0.32 | 100 | Not applicable | |
| J-19 | 0.04 | 100 | | |

### 3.2.5 Human PBMC Activation Assay

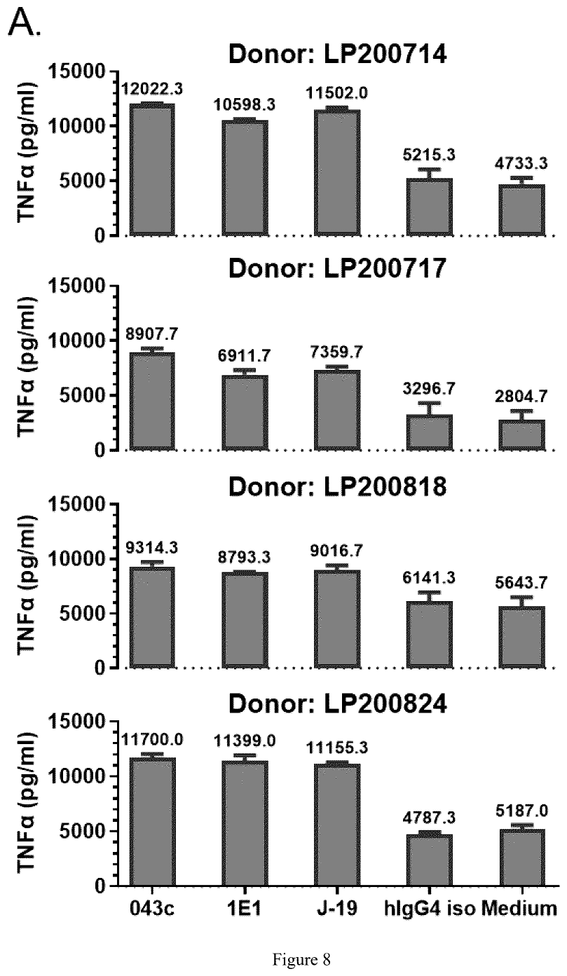

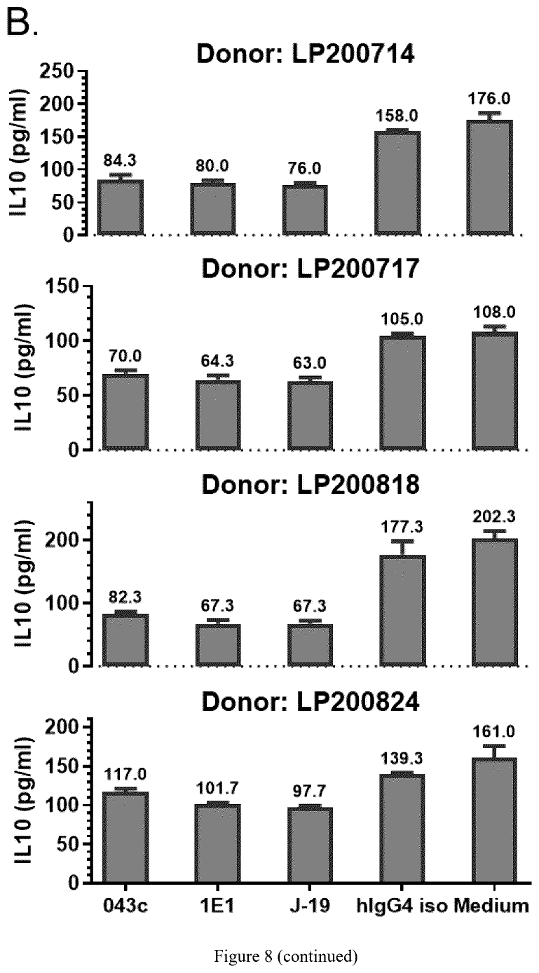

The effects of 043c, 1E1 and J-19 on PBMC activation were studied. Briefly, peripheral blood mononuclear cells (PBMCs) collected from healthy human donors (Donor #LP200714, LP200717, LP200818, LP200824) were preincubated with 6.67 μg/ml anti-LILRB2 antibodies or hIgG4 isotype control at 37° C. overnight and then primed by LPS (2 ng/ml) for 24 hours. Then the released TNFα (FIG. 8A) and IL-10 (FIG. 8B) in harvested supernatants were quantified by HTRF kits (Cisbio). 043c, 1E1 and J-19 upregulated the release of pro-inflammatory cytokine TNFα, while downregulated the release of anti-inflammatory cytokine IL-10, indicating that blocking LILRB2 with 043c promoted LPS-primed human PBMCs into a pro-inflammation status.

### 3.2.6 Human Monocyte Activation Assay

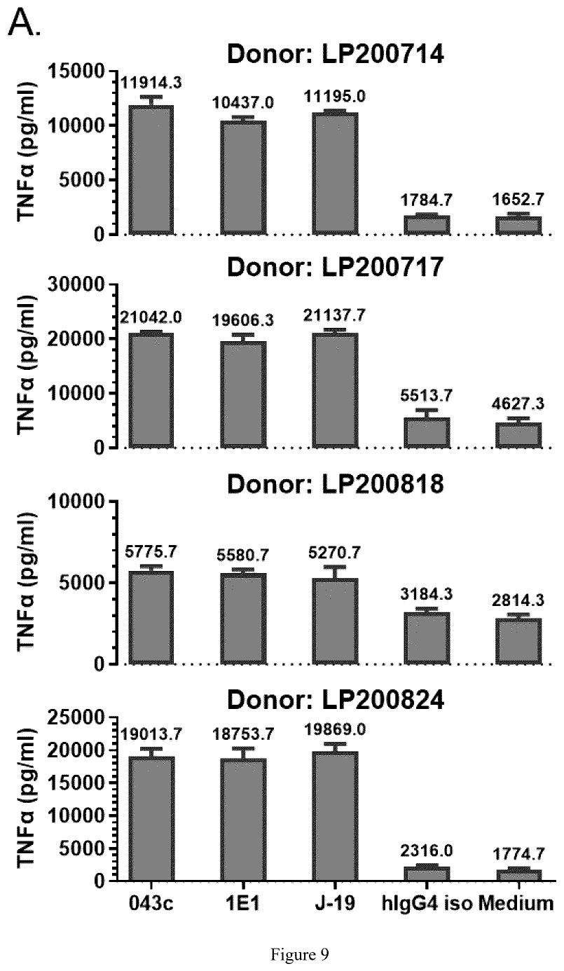

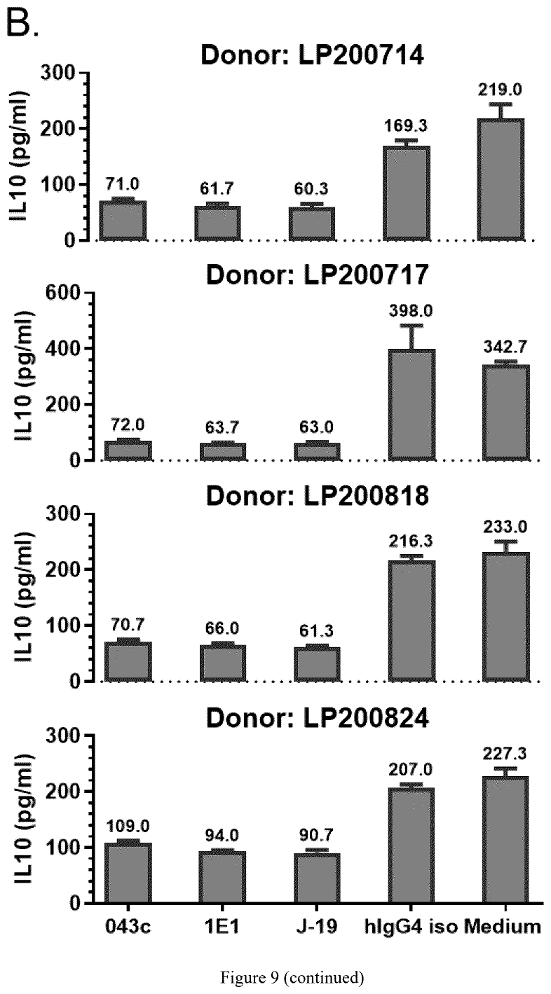

The effects of 043c, 1E1 and J-19 on monocyte activation were also studied. Briefly, human monocytes isolated from PBMCs of healthy donors (Donor #LP200714, LP200717, LP200818, LP200824) were preincubated with 6.67 μg/ml anti-LILRB2 antibodies or hIgG4 isotype control at 37° C. overnight and then primed by LPS (2 ng/ml) for 24 hours. Then the released TNFα (FIG. 9A) and IL-10 (FIG. 9B) in harvested supernatants were quantified by HTRF kits (Cisbio). Similar as the results obtained in PBMC activation assay, 043c, 1E1 and J-19 upregulated the release of pro-inflammatory cytokine TNFα, while downregulated the release of anti-inflammatory cytokine IL-10, indicating that blocking LILRB2 with 043c promoted LPS-primed human monocytes into a pro-inflammation status.

### 3.2.7 Macrophage Polarization Assay

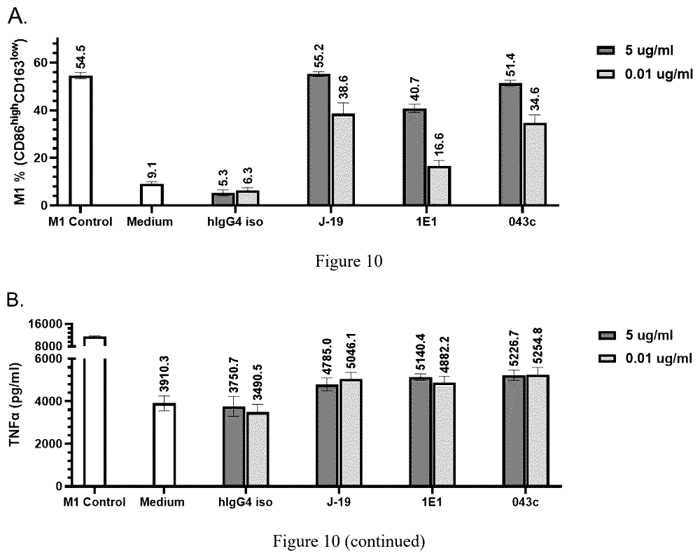

The effects of 043c, 1E1 and J-19 on macrophage differentiation were studied with macrophage polarization assay. Briefly, in the presence of anti-LILRB2 antibodies or hIgG4 isotype control, human monocytes isolated from PBMCs of healthy donors were treated with M-CSF (colony stimulating factor 1) for 5 days and then polarized with IL-4 and IL13 for another 2 days, which are usually used for anti-inflammation M2 macrophage polarization. At the end of the study, the effects of the tested antibodies on macrophage phenotype switch were determined by quantitating the percentage of CD86^{high}CD163^{low} population (M1%) and the release of TNFα. Human monocytes were also directly polarized into pro-inflammation M1 macrophages using GM-CSF and IFN γ as system control (M1 control). Similar as M1 control, 043c, 1E1 and J-19 potently upregulated M1 macrophage population (FIG. 10A) and the release of pro-inflammatory cytokine TNFα (FIG. 10B), indicating that blocking LILRB2 with 043c reprogramed human monocyte derived M2 macrophages into pro-inflammation M1 phenotype.

### 3.2.8 M2-T Co-Culture Assay

The effects of 043c, 1E1 and J-19 to relieve M2 macrophages mediated suppression of T cells were studied with M2 macrophage-T cell (M2-T) co-culture assay. Briefly, in the presence of 5 μg/ml anti-LILRB2 antibodies or hIgG4 isotype control, human monocytes isolated from PBMCs of healthy donors were treated with M-CSF for 5 days and then polarized toward anti-inflammation M2 phenotype with IL-4 and IL13 for another 2 days. The monocyte derived macrophages differentiated with or without anti-LILRB2 antibodies were mixed with autologous T cells at various ratios of 1:0.5, 1:1, 1:2, 1:4 and 1:8 in the presence of 10 ng/ml OKT3 (murine monoclonal antibody of the immunoglobulin IgG2a isotype). The released IL-2 in supernatant harvested after 48 hours of co-culture, IFNγ and TNFα in supernatants harvested after 120 hours of co-culture were quantified by HTRF kits (Cisbio).

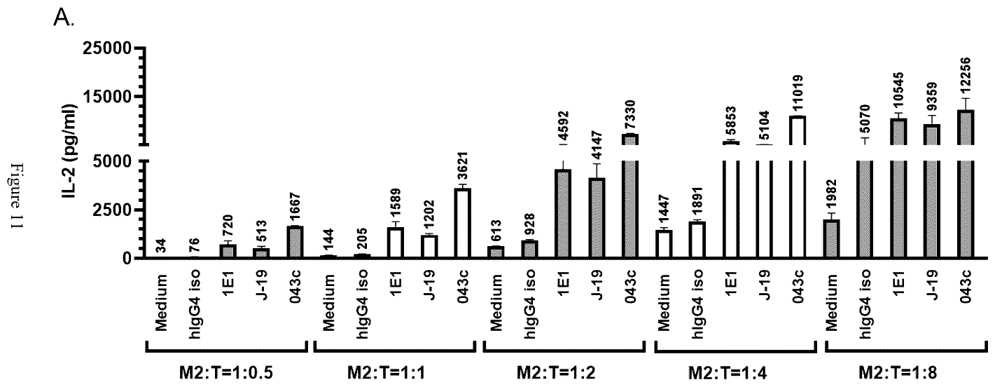

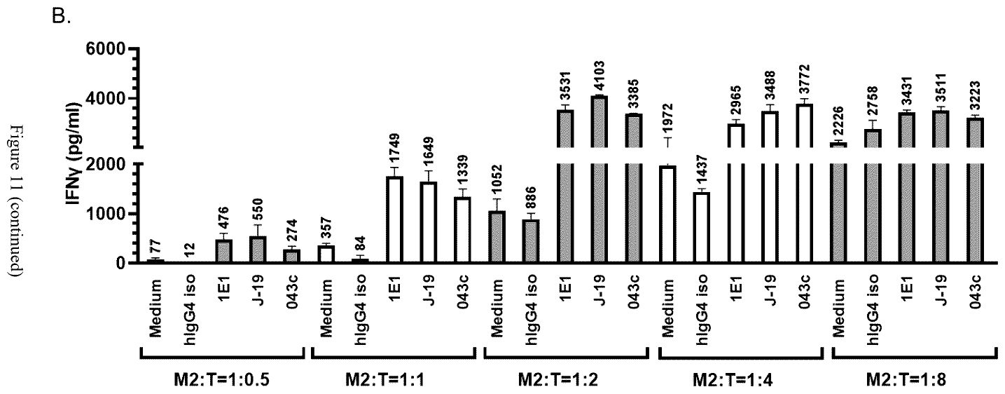

At all M2-T mixing ratios, 043c, 1E1 and J-19 potently upregulated the release of IL-2 (FIG. 11A), IFNγ (FIG. 11B) and TNFα (FIG. 11C). The supernatants harvested after 120 hours of equal proportional co-culture (M2-T mixing ratio=1:1) were also assayed for cytokine/chemokine release profile by Luminex. As shown in Table 6, more pro-inflammation cytokines and chemokines such as Granzyme B, IL-3, IL-5 and CCL4 were found upregulated under the treatment of 043c. These results indicated that blocking LILRB2 with 043c relieved the suppressive function of M2 macrophages.

TABLE 6
Cytokine/chemokine release in M2-T coculture assay

| | Medium | Relative cytokine/chemokine level | | | | |
|---|---|---|---|---|---|---|
| Cytokine | (pg/ml) | Medium | hIgG4 iso | 1E1 | J-19 | 043c |
| IL-2 | 72.4 | 1.0 | 1.6 | 8.2 | 5.2 | 17.8 |
| IFN-gamma | 115.1 | 1.0 | 0.5 | 4.6 | 4.5 | 4.1 |
| Granzyme B | 183.3 | 1.0 | 0.7 | 4.6 | 5.0 | 7.6 |
| TNF-alpha | 542.7 | 1.0 | 1.1 | 4.3 | 4.1 | 4.8 |
| PDGF-AA | 66.6 | 1.0 | 1.2 | 3.3 | 3.1 | 4.3 |
| IL-3 | 279.6 | 1.0 | 1.4 | 3.0 | 3.0 | 3.6 |
| IL-1beta/IL-1F2 | 6.6 | 1.0 | 0.9 | 2.6 | 2.2 | 1.9 |
| IL-5 | 14.4 | 1.0 | 1.1 | 2.5 | 3.3 | 2.3 |
| TGF-alpha | 47.8 | 1.0 | 1.0 | 2.1 | 2.1 | 3.2 |
| IFN-alpha | 4.1 | 1.0 | 1.1 | 1.7 | 1.6 | 2.2 |
| CCL4/MIP-1 beta | 536.2 | 1.0 | 1.0 | 1.7 | 1.7 | 2.4 |
| FGF-basic | 14.2 | 1.0 | 1.0 | 1.6 | 1.2 | 1.3 |
| IL-13 | 59.7 | 1.0 | 1.0 | 1.6 | 1.8 | 1.8 |
| G-CSF | 9.8 | 1.0 | 1.0 | 1.6 | 1.6 | 2.3 |
| IL-4 | 3.0 | 1.0 | 1.1 | 1.5 | 1.6 | 1.6 |
| B7-H1/PD-L1 | 289.2 | 1.0 | 0.9 | 1.5 | 1.5 | 1.8 |
| CCL20/MIP-3 alpha | 18.2 | 1.0 | 0.8 | 1.4 | 1.4 | 2.2 |
| IL-15 | 14.3 | 1.0 | 0.8 | 1.4 | 1.3 | 1.8 |
| GM-CSF | 457.7 | 1.0 | 1.0 | 1.3 | 1.4 | 1.6 |
| CCL5/RANTES | 75.0 | 1.0 | 1.2 | 1.3 | 1.4 | 3.8 |
| CCL3/MIP-1 alpha | 186.2 | 1.0 | 1.1 | 1.3 | 1.2 | 1.3 |
| PDGF-AB/BB | 10.6 | 1.0 | 0.9 | 1.1 | 1.0 | 1.5 |
| CCL19/MIP-3 beta | 205.0 | 1.0 | 0.7 | 0.7 | 0.7 | 0.3 |
| CXCL2/GRO beta | 712.4 | 1.0 | 0.8 | 0.5 | 0.5 | 0.4 |
| IL-6 | 10200.5 | 1.0 | 1.1 | 0.7 | 0.6 | 0.2 |
| VEGF | 350.4 | 1.0 | 0.8 | 1.1 | 1.0 | 0.3 |

### 3.2.9 DC Differentiation Assay

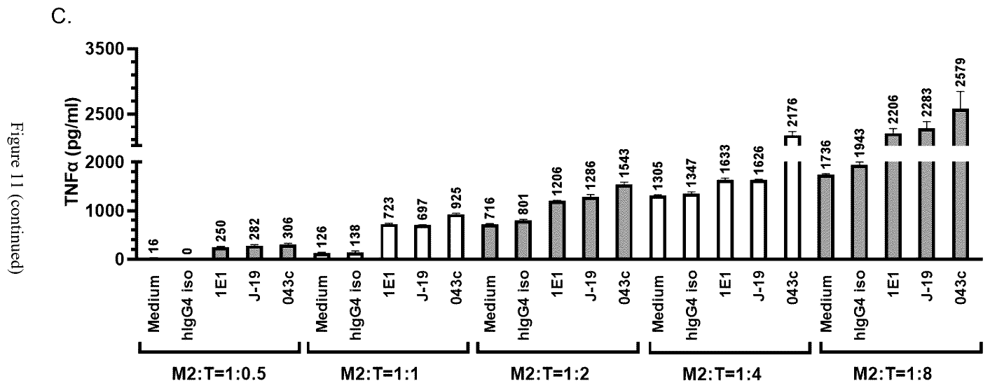

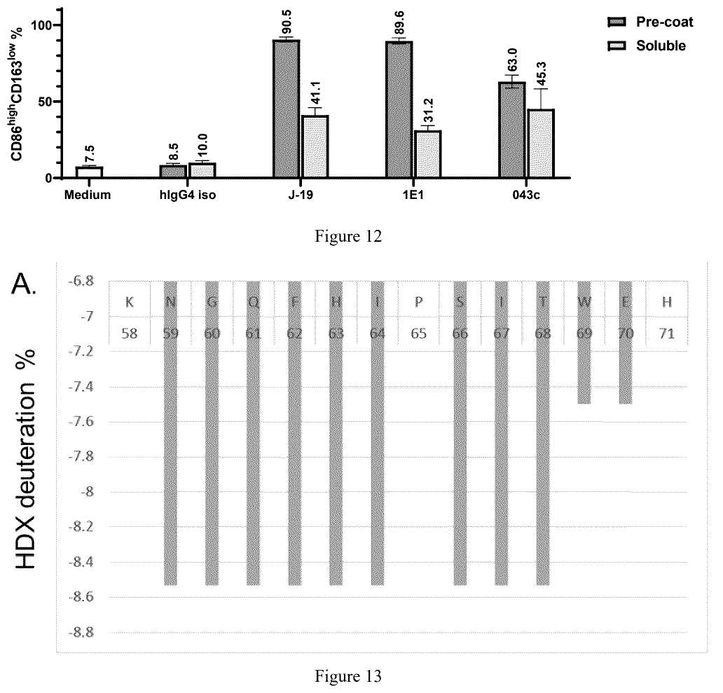

The effects of 043c, 1E1 and J-19 on dendritic cells (DC) maturation were studied with DC differentiation assay. Briefly, human monocytes isolated from PBMCs of healthy donors were treated with GM-CSF and IL-4 for six days to differentiate into immature DC cells. The monocytes derived immature DC cells were then incubated with plate coated or soluble anti-LILRB2 antibodies (5 μg/ml) for another two days followed by a prime by LPS (10 ng/ml) for 6 hours. At the end of the study, the effects of the tested antibodies on dendritic cells maturation were determined by quantitating the percentage of CD86^{high}CD163^{low} population by FACS, and the release of pro-inflammation cytokines and chemokines by Luminex. As shown in FIG. 12 and Table 7, 043c, 1E1 and J-19 potently upregulated CD86^{high}CD163^{low} DC population, and the release of pro-inflammation cytokines and chemokines such as TNFα, IL-6, IFN-beta and CCL3, indicating that blocking LILRB2 with 043c promoted immature DCs into a maturation status.

TABLE 7
Cytokine/chemokine release in DC differentiation assay

| | | Relative cytokine level | | | | |
|---|---|---|---|---|---|---|
| Treatment | | | hIgG4 | | | |
| format | Cytokine | Medium | iso | J-19 | 1E1 | 043c |
| Pre-coat | TNFα | 0.83 | 1.00 | 2.13 | 2.33 | 5.38 |
| | IL-6 | 0.95 | 1.00 | 0.50 | 0.53 | 1.61 |
| | IFN-beta | 0.78 | 1.00 | 0.24 | 0.26 | 3.39 |
| | IL-10 | 0.92 | 1.00 | 0.86 | 1.16 | 2.16 |
| | VEGF | 1.24 | 1.00 | 1.43 | 1.57 | 1.67 |
| | CCL20/MIP-3 alpha | 0.93 | 1.00 | 1.37 | 1.33 | 1.75 |
| | CCL3/MIP-1 alpha | 0.79 | 1.00 | 3.92 | 2.05 | >5 |
| | CCL22/MDC | 0.92 | 1.00 | 20.92 | 17.41 | 2.61 |
| | IL-1 alpha/lL-1F1 | 0.95 | 1.00 | 1.81 | 1.95 | 1.71 |
| | CCL23/MPIF-1 | 1.02 | 1.00 | 0.25 | 0.25 | 0.48 |
| Soluble | TNFα | 1.19 | 1.00 | 4.43 | 3.81 | 4.71 |
| | IL6 | 1.08 | 1.00 | 1.69 | 1.67 | 1.93 |
| | IL-8/CXCL8 | 1.11 | 1.00 | 1.45 | 1.47 | 1.65 |
| | IFN-beta | 1.24 | 1.00 | 4.18 | 5.03 | 7.24 |
| | IL-10 | 1.05 | 1.00 | 1.51 | 1.59 | 1.69 |
| | CCL2/JE/MCP-1 | 1.07 | 1.00 | 1.84 | 1.85 | 2.36 |
| | VEGF | 0.90 | 1.00 | 1.48 | 1.29 | 1.43 |
| | CCL20/MIP-3 alpha | 1.12 | 1.00 | 1.81 | 1.76 | 1.88 |
| | CCL3/MIP-1 alpha | 1.11 | 1.00 | 4.36 | 3.56 | 5.79 |
| | CCL22/MDC | 1.06 | 1.00 | 2.41 | 1.70 | 2.03 |
| | IL-1 alpha/lL-1F1 | 1.07 | 1.00 | 1.71 | 1.57 | 1.71 |
| | CCL23/MPIF-1 | 0.94 | 1.00 | 0.57 | 0.69 | 0.73 |

### 3.2.10 Epitope Analysis

The epitope binning for 043c, 1E1 and J19 was carried out by octet. Briefly, the HIS1K sensors were hydrolyzed for 10 minutes followed by capture of 6×His-tagged human LILRB2 ECD recombinant protein to 0.3 nm. The LILRB2 captured sensors were then exposed to Ab1 and Ab2 in tandem or Ab2 directly. The binding signals of Ab2 to LILRB2 under different conditions were detected respectively. Finally, the relative binding signals of Ab2 in tandem were calculated by normalization. High relative binding signals represent low competition between Ab2 and Ab1 for binding antigen. Likewise, low relative association signals represent high competition between Ab2 and Ab1. As shown in Table 8, J-19 and 1E1 competed for binding to LILRB2, while 043c had no competition with J-19 and 1E1.

TABLE 8
Epitope binning of 043c, 1E1 and J-19

| | Ab2 | | |
|---|---|---|---|
| Ab1 | J-19 | 1E1 | 043c |
| J-19 | −3% | 17% | 100% |
| 1E1 | 32% | 3% | 108% |
| 043c | 96% | 100% | 3% |

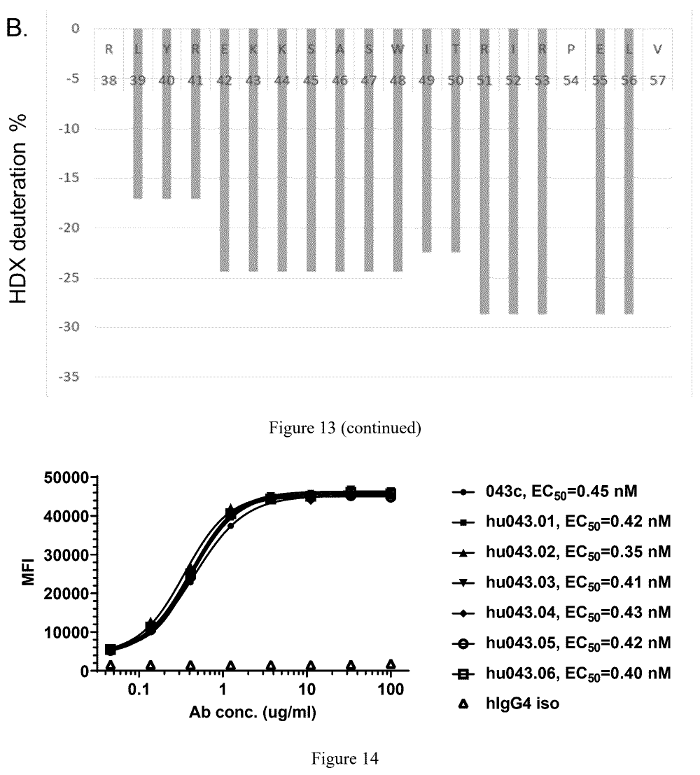

The binding epitopes of 043c and 1E1 on LILRB2 were further mapped using hydrogen deuterium exchange mass spectrometry (HDX-MS). The binding of 043c resulted in reduced hydrogen deuterium exchange in the peptide “NGQFHIPSITWE” (binding site 1, SEQ ID NO: 21), indicating that this region on LILRB2 is critical for 043c to bind (FIG. 13A). Interestingly the binding of 1E1 resulted in reduced hydrogen deuterium exchange in a different peptide “LYREKKSASWITRIRPEL” (binding site 2, SEQ ID NO: 22) (FIG. 13B).

Taking the results obtained using Octet and HDX-MS together, it can be concluded that 043c has a unique binding epitope distinct from 1E1 and J-19.

### Example 4. Antibody Humanization

### 4.1 Humanization Design

Complementarity-determining region (CDR) grafting method was used for humanization of 043c. Briefly, IGHV1-46*01 (IMGT allele name) and IGKV1-33*01 (IMGT allele name) were first selected as humanization templates for heavy chain and light chain, respectively, based on their homology to the original mouse antibody sequences. CDRs were defined using Kabat definition except heavy chain CDR1, which was defined using a combination of Kabat and Chothia systems. For grafting, the CDRs and different combinations of canonical residues from 043c were grafted onto the templates. The resulting variants (human IgG4 antibodies with S228P mutation in the constant region) were produced and designated as hu043.01 to hu043.08, where the prefix “hu” indicates “humanized”, and the number in the suffix denotes the serial number. All the variants were tested in multiple in vitro assays to select the best ones that retained the property of the parental antibody.

### 4.2 Characterization of the Humanized Variants

### 4.2.1 Binding Activity

The binding of 043c and 043c derived humanized variants to cell membrane human LILRB2 was detected by FACS assay using EL4/LILRB2 cells. As shown in FIG. 14, hu043.01, hu043.02, hu043.03, hu043.04, hu043.05 and hu043.06 retained the same human LILRB2 binding activity as the parental antibody 043c. The EC₅₀ and top MFI values analyzed using Four-Parameter nonlinear fitting by GraphPad Prism 9.0 are summarized in Table 9.

TABLE 9
Binding of 043c and 043c derived humanized variants to EL4/LILRB2

| Ab code | 043c | hu043.01 | hu043.02 | hu043.03 | hu043.04 | hu043.05 | hu043.06 |
|---|---|---|---|---|---|---|---|
| EC₅₀ (nM) | 0.45 | 0.42 | 0.35 | 0.41 | 0.43 | 0.42 | 0.40 |
| TOP MFI | 45273 | 45828 | 45663 | 46324 | 45027 | 45096 | 45722 |

### 4.2.2 Affinity Detection

The binding affinity of 043c derived humanized variants to human LILRB2 was determined using Bio-Layer Interferometry (Octet) or Surface Plasmon Resonance (Biacore). The association and dissociation curves were fitted with 1:1 binding model, and the Ka/Kd/KD values for each antibody were calculated and summarized in Table 10. hu043.01, hu043.02, hu043.03, hu043.04, hu043.05, hu043.06, hu043.07 and hu043.08 retained the same human LILRB2 binding affinity as the parental antibody 043c.

TABLE 10
Human LILRB2 binding affinity of
043c derived humanized variants

| Detection | | | | |
|---|---|---|---|---|
| approach | Ab code | KD (nM) | kd (1/s) | ka (1/Ms) |
| Octet | 043c | 2.40 | 4.94E+05 | 1.19E−03 |
| | hu043.01 | 3.19 | 5.16E+05 | 1.64E−03 |
| | hu043.02 | 3.57 | 5.17E+05 | 1.84E−03 |
| | hu043.03 | 5.22 | 4.66E+05 | 2.43E−03 |
| | hu043.04 | 3.78 | 4.88E+05 | 1.85E−03 |
| | hu043.05 | 3.90 | 5.01E+05 | 1.99E−03 |
| | hu043.06 | 3.63 | 5.12E+05 | 1.86E−03 |
| Biacore | hu043.07 | 1.68 | 2.25E+06 | 3.79E−03 |
| | hu043.08 | 1.71 | 2.21E+06 | 3.78E−03 |

### 4.2.3 Blocking Activity

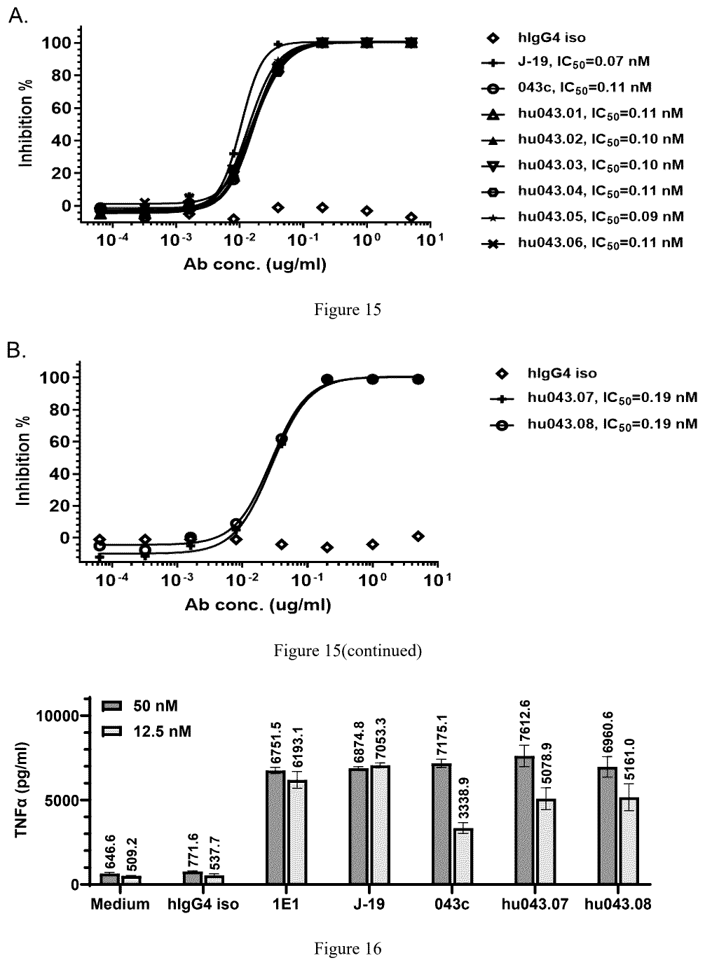

The activity of 043c and 043c derived humanized variants to block the interaction between LILRB2 and HLA-G was assessed by competitive FACS (FIG. 15A and FIG. 15B, refer to the method described in example 3.2.4). The IC₅₀ values and top blocking ratios analyzed using Four-Parameter nonlinear fitting by GraphPad Prism 9.0 are summarized in Table 11. hu043.01, hu043.02, hu043.03, hu043.04, hu043.05, hu043.06, hu043.07 and hu043.08 retained the same LILRB2/HLA-G interaction blocking activity as the parental antibody 043c.

TABLE 11
Blocking activity of 043c and 043c derived humanized variants

LILRB2/HLA-G interaction blockade

| Ab code | IC₅₀ (nM) | TOP Blocking (%) |
|---|---|---|
| J-19 | 0.07 | 100 |
| 043c | 0.11 | 100 |
| hu043.01 | 0.11 | 100 |
| hu043.02 | 0.10 | 100 |
| hu043.03 | 0.10 | 100 |
| hu043.04 | 0.11 | 100 |
| hu043.05 | 0.09 | 100 |
| hu043.06 | 0.11 | 100 |
| hu043.07 | 0.19 | 100 |
| hu043.08 | 0.19 | 100 |

### 4.2.4 Function Validation

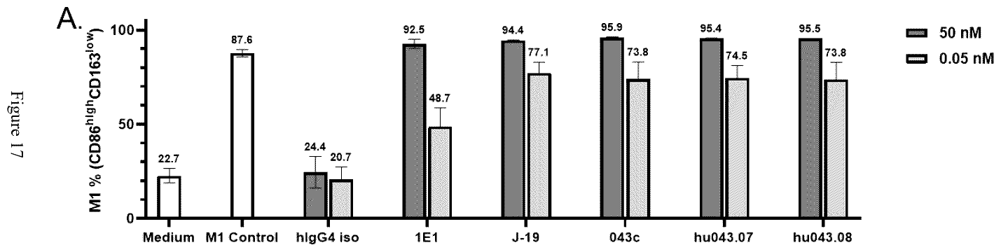

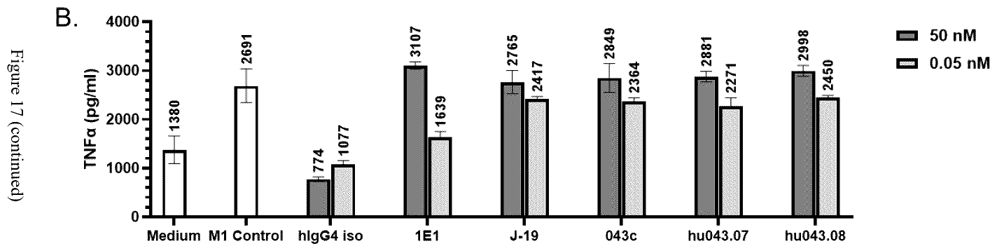

The function of hu043.07 and hu043.08 were validated using monocyte activation assay (refer to the method described in example 3.2.6) and macrophage polarization assay (refer to the method described in example 3.2.7). hu043.07 and hu043.08 upregulated the release of pro-inflammatory cytokine TNFα in monocyte activation assay (FIG. 16). They also potently upregulated M1 macrophage population (FIG. 17A) and the release of anti-inflammatory cytokine TNFα (FIG. 17B). In both studies, hu043.07 and hu043.08 exhibited similar potency to 1E1, J-19 and the parental antibody 043c.

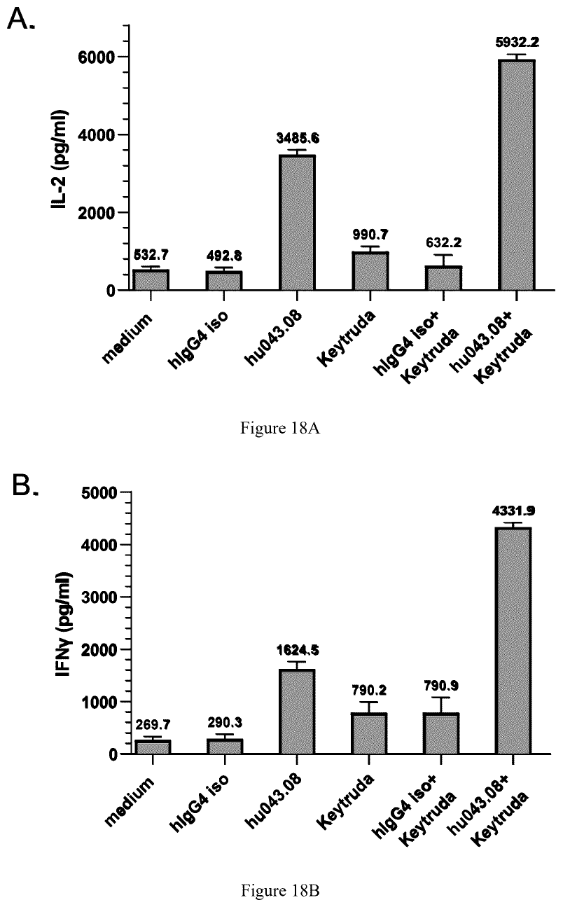

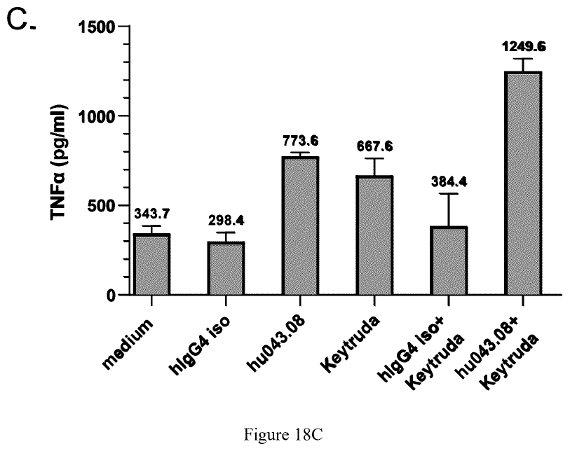

The function of hu043.08 or hu043.08 in combination with Keytruda (anti-PD-1) was further explored using M2-T co-culture assay. Briefly, in the presence of 50 nM hu043.08 or hIgG4 isotype control, human monocytes isolated from PBMCs of healthy donors were treated with M-CSF for 5 days and then polarized toward anti-inflammation M2 phenotype with IL-4 and IL13 for another 2 days. The monocyte derived macrophages differentiated with or without hu043.08 were mixed with autologous T cells at the ratio of 1:1 in the presence of 10 ng/ml OKT3 with or without 5 nM Keytruda. The released IL-2 in supernatant harvested after 24 hours of co-culture, IFNγ and TNFα in supernatants harvested after 96 hours of co-culture were quantified by HTRF kits (Cisbio). Hu043.08 potently upregulated the release of IL-2 (FIG. 18A), IFNγ (FIG. 18B) and TNFα (FIG. 18C) in the test. While Keytruda alone only mildly upregulated the release of these pro-inflammation cytokines (FIG. 18). Interestingly the combination of hu043.08 with Keytruda induced greater release of these pro-inflammation cytokines in the test (FIG. 18). These results suggest that hu043.08 relieved the suppressive function of M2 macrophages and hu043.08 synergized with anti-PD-1 antibody to reinvigorate T cell functionalities.

Exemplary heavy chain variable regions and light chain variable regions of 043c derived humanized antibodies, i.e., hu043.01, hu043.02, hu043.03, hu043.04, hu043.05, hu043.06, hu043.07 and hu043.08, are as shown in table 12.

TABLE 12
VL and VH of 043c derived humanized antibodies

| ID | Heavy chain ID | Light chain ID |
|---|---|---|
| hu043.01 | hu043VH6 | hu043VL4 |
| hu043.02 | hu043VH7 | hu043VL4 |
| hu043.03 | hu043VH8 | hu043VL4 |
| hu043.04 | hu043VH6 | hu043VL2 |
| hu043.05 | hu043VH7 | hu043VL2 |
| hu043.06 | hu043VH8 | hu043VL2 |
| hu043.07 | hu043VH9 | hu043VL1 |
| hu043.08 | hu043VH10 | hu043VL1 |

* The prefix “hu” indicates “humanized”, “VH” indicates “heavy chain variable regions”, “VL” indicates “light chain variable regions”, and the number in the suffix denotes the serial number.

The amino acid sequences in the disclosure are listed in Table 13. The CDRs of VH and VL are underlined, and CDRs are defined using Kabat definition.

TABLE 13
Amino acid Sequences

| Sequence ID No. | Name | Amino acid sequence (single-letter code) |
|---|---|---|
| SEQ ID NO: 1 | VH CDR1 | SYWMY |
| SEQ ID NO: 2 | VH CDR2 | HINPSSGYTKYNQKFKD |
| SEQ ID NO: 3 | VH CDR3 | DPNWDNY |
| SEQ ID NO: 4 | VL CDR1 | KASQDVSIAVA |
| SEQ ID NO: 5 | VL CDR2 | SASYRYT |
| SEQ ID NO: 6 | VL CDRL3 | QQHYSTPYT |
| SEQ ID NO: 7 | hu043 VH6 | QVQLVQSGAEVKKPGASVKVSCKASGYTFTSYWMYW |
| | | VRQAPGQGLEWIGHINPSSGYTKYNQKFKDRATLTADT |
| | | STSTAYMELSSLRSEDTAVYYCARDPNWDNYWGQGTT |
| | | VTVSS |
| SEQ ID NO: 8 | hu043 VH7 | QVQLVQSGAEVKKPGASVKVSCKASGYTFTSYWMYW |
| | | VRQAPGQGLEWIGHINPSSGYTKYNQKFKDRVTMTAD |
| | | TSTSTVYMELSSLRSEDTAVYYCARDPNWDNYWGQG |
| | | TTVTVSS |
| SEQ ID NO: 9 | hu043 VH8 | QVQLQQSGAEVKKPGASVKLSCKASGYTFTSYWMYW |
| | | VRQRPGQGLEWIGHINPSSGYTKYNQKFKDRATLTADT |
| | | STSTAYMELSSLRSEDTAVYYCARDPNWDNYWGQGTT |
| | | VTVSS |
| SEQ ID NO: 10 | hu043 VH9 | QVQLVQSGAEVKKPGASVKVSCKASGYTFTSYWMYW |
| | | VRQAPGQGLEWIGHINPSSGYTKYNQKFKDKATLTAD |
| | | KSTSTAYMELSSLRSEDTAVYYCARDPNWDNYWGQGT |
| | | TVTVSS |
| SEQ ID NO: 11 | hu043VH10 | QVQLVQSGAEVKKPGASVKVSCKASGYTFTSYWMYW |
| | | VRQAPGQGLEWIGHINPSSGYTKYNQKFKDKATLTAD |
| | | KSTSTAYMELSSLRSEDTAVYYCARDPNWDNYWGQGT |
| | | TLTVSS |
| SEQ ID NO: 12 | Hybridoma/0 | QVQLQQSGAELAKPGASVKLSCKASGYTFTSYWMYW |
| | 43cVH | VKQRPGQGLEWIGHINPSSGYTKYNQKFKDKATLTAD |
| | | KSSSTAYMQLSSLTYEDSAVYYCARDPNWDNYWGQG |
| | | TTLTVSS |
| SEQ ID NO: 13 | hu043 VL4 | DIVMTQSPSSLSASVGDRVSITCKASQDVSIAVAWYQQ |
| | | KPGKAPKLLIYSASYRYTGVPDRFSGSGSGTDFTFTISS |
| | | LQPEDIATYYCQQHYSTPYTFGGGTKLEIK |
| SEQ ID NO: 14 | hu043 VL2 | DIQMTQSPSSLSASVGDRVTITCKASQDVSIAVAWYQQ |
| | | KPGKAPKLLIYSASYRYTGVPSRFSGSGSGTDFTFTISSL |
| | | QPEDIATYYCQQHYSTPYTFGGGTKLEIK |
| SEQ ID NO: 15 | hu043VL1 | DIQMTQSPSSMSASVGDRVTITCKASQDVSIAVAWYQQ |
| | | KPGKAPKLLIYSASYRYTGVPSRFSGSGSGTDFTFTISS |
| | | VQPEDIATYYCQQHYSTPYTFGGGTKLEIK |
| SEQ ID NO: 16 | Hybridoma/ | DIVMTQSHKFMSTSVGDRVSITCKASQDVSIAVAWYQQ |
| | 043cVL | KPGQSPKLLIYSASYRYTGVPDRFTGSGSGTDFTFTISS |
| | | VQAEDLAVYYCQQHYSTPYTFGGGTKLEIK |
| SEQ ID NO: 23 | Wild-type | MTPIVTVLICLGLSLGPRTRVQTGTIPKPTLWAEPDSVIT |
| | human | QGSPVTLSCQGSLEAQEYRLYREKKSASWITRIRPELVK |
| | LILRB2 | NGQFHIPSITWEHTGRYGCQYYSRARWSELSDPLVLVM |
| | | TGAYPKPTLSAQPSPVVTSGGRVTLQCESQVAFGGFILC |
| | | KEGEDEHPQCLNSQPHARGSSRAIFSVGPVSPNRRWSH |
| | | RCYGYDLNSPYVWSSPSDLLELLVPGVSKKPSLSVQPG |
| | | PVMAPGESLTLQCVSDVGYDRFVLYKEGERDLRQLPG |
| | | RQPQAGLSQANFTLGPVSRSYGGQYRCYGAHNLSSEC |
| | | SAPSDPLDILITGQIRGTPFISVQPGPTVASGENVTLLCQ |
| | | SWRQFHTFLLTKAGAADAPLRLRSIHEYPKYQAEFPMS |
| | | PVTSAHAGTYRCYGSLNSDPYLLSHPSEPLELVVSGPS |
| | | MGSSPPPTGPISTPGPEDQPLTPTGSDPQSGLGRHLGVVI |
| | | GILVAVVLLLLLLLLLFLILRHRRQGKHWTSTQRKADF |
| | | QHPAGAVGPEPTDRGLQWRSSPAADAQEENLYAAVKD |
| | | TQPEDGVEMDTRAAASEAPQDVTYAQLHSLTLRRKAT |
| | | EPPPSQEREPPAEPSIYATLAIH |

* The prefix “hu” indicates “humanized”, “VH” indicates “heavy chain variable regions”, “VL” indicates “light chain variable regions”, “H” indicates “heavy chain”, “L” indicates “light chain”, “CDR” indicates “complementarity determining region”, and the number in the suffix denotes the serial number.

The amino acid is represented as standard single-letter code according to the standard IUPAC (International Union of Pure and Applied Chemistry) amino acid abbreviation.

While certain embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications, variations, improvements, and substantial equivalents.

1. An antibody or antigen-binding fragment thereof that binds to LILRB2 (Leukocyte Immunoglobulin Like Receptor B2) comprising:
a heavy chain variable region (VH) comprising complementarity determining regions (CDRs) 1, 2, and 3, wherein the VH CDR1 comprises an amino acid sequence that is at least 80%, 85%, 90%, 95%, 96%, 97%, 98%, 99% identical to the sequence set forth in SEQ ID NO: 1; the VH CDR2 comprises an amino acid sequence that is at least 80%, 85%, 90%, 95%, 96%, 97%, 98%, 99% identical to the sequence set forth in SEQ ID NO: 2; the VH CDR3 comprises an amino acid sequence that is at least 80%, 85%, 90%, 95%, 96%, 97%, 98%, 99% identical to the sequence set forth in SEQ ID NO: 3; and
a light chain variable region (VL) comprising CDRs 1, 2, and 3, wherein the VL CDR1 comprises an amino acid sequence that is at least 80%, 85%, 90%, 95%, 96%, 97%, 98%, 99% identical to the sequence set forth in SEQ ID NO: 4; the VL CDR2 comprises an amino acid sequence that is at least 80%, 85%, 90%, 95%, 96%, 97%, 98%, 99% identical to the sequence set forth in SEQ ID NO: 5; the VL CDR3 comprises an amino acid sequence that is at least 80%, 85%, 90%, 95%, 96%, 97%, 98%, 99% identical to the sequence set forth in SEQ ID NO: 6;
optionally,
the VH comprises CDRs 1, 2, and 3 with the amino acid sequences set forth in SEQ ID NOs: 1, 2, 3, respectively,
and the VL comprises CDRs 1, 2, and 3 with the amino acid sequences set forth in SEQ ID NOs: 4, 5, 6, respectively.

2. (canceled)

3. The antibody or antigen-binding fragment thereof of claim 1, wherein the VH is at least 80%, 85%, 86%, 87%, 88%, 89%, 90%, 91%, 92%, 93%, 94%, 95%, 96%, 97%, 98%, 99% identical to the amino acid sequence set forth in SEQ ID NO: 7, 8, 9, 10, 11 or 12; and
the VL is at least 80%, 85%, 86%, 87%, 88%, 89%, 90%, 91%, 92%, 93%, 94%, 95%, 96%, 97%, 98%, 99% identical to the amino acid sequence set forth in SEQ ID NO: 13, 14, 15 or 16;
optionally,
the VH is different from the amino acid sequence set forth in SEQ ID NO: 7, 8, 9, 10, 11 or 12 by no more than 25, 20, 15, 13, 10, 9, 8, 7, 6, 5, 4, 3, 2 or 1 amino acid; and
the VL is different from the amino acid sequence set forth in SEQ ID NO: 13, 14, 15 or 16 by no more than 20, 15, 13, 10, 9, 8, 7, 6, 5, 4, 3, 2 or 1 amino acid;
optionally,
the VH comprises the amino acid sequence set forth in SEQ ID NO: 7, 8, 9, 10, 11 or 12; and
the VL comprises the amino acid sequence set forth in SEQ ID NO: 13, 14, 15 or 16.

4. (canceled)

5. (canceled)

6. The antibody or antigen-binding fragment thereof of claim 1, wherein the VH comprises the amino acid sequence of SEQ ID NO: 7 and the VL comprises the amino acid sequence of SEQ ID NO: 13,
the VH comprises the amino acid sequence of SEQ ID NO: 8 and the VL comprises the amino acid sequence of SEQ ID NO: 13,
the VH comprises the amino acid sequence of SEQ ID NO: 9 and the VL comprises the amino acid sequence of SEQ ID NO: 13,
the VH comprises the amino acid sequence of SEQ ID NO: 7 and the VL comprises the amino acid sequence of SEQ ID NO: 14,
the VH comprises the amino acid sequence of SEQ ID NO: 8 and the VL comprises the amino acid sequence of SEQ ID NO: 14,
the VH comprises the amino acid sequence of SEQ ID NO: 9 and the VL comprises the amino acid sequence of SEQ ID NO: 14,
the VH comprises the amino acid sequence of SEQ ID NO: 10 and the VL comprises the amino acid sequence of SEQ ID NO: 15;
the VH comprises the amino acid sequence of SEQ ID NO: 11 and the VL comprises the amino acid sequence of SEQ ID NO: 15; or
the VH comprises the amino acid sequence of SEQ ID NO: 12 and the VL comprises the amino acid sequence of SEQ ID NO: 16.

7. The antibody or antigen-binding fragment thereof of claim 1, wherein the antibody or antigen-binding fragment thereof specifically binds to LILRB2, optionally,
the antibody or antigen-binding fragment thereof specifically binds to LILRB2 with an EC₅₀ value of less than 20 nM, less than 10 nM, less than 5 nM or less than 1 nM;
the antibody or antigen-binding fragment thereof blocks the interaction between human LILRB2 and HLA-A2;
optionally, the antibody or antigen-binding fragment thereof blocks the interaction between human LILRB2 and HLA-A2 with an IC₅₀ value of less than 20 nM, less than 10 nM, less than 5 nM, less than 1 nM, or less than 0.6 nM;
the antibody or antigen-binding fragment thereof blocks the interaction between human LILRB2 and HLA-G;
optionally,
the antibody or antigen-binding fragment thereof blocks the interaction between human LILRB2 and HLA-G with an IC₅₀ value of less than 10 nM, less than 5 nM, less than 1 nM, less than 0.5 nM, or less than 0.3 nM; or
the antibody or antigen-binding fragment thereof binds human LILRB2 with KD (affinity constant) of less than 15 nM, less than 10 nM, less than 8 nM, or less than 6 nM.

8. (canceled)

9. (canceled)

10. (canceled)

11. (canceled)

12. The antibody or antigen-binding fragment thereof of claim 1, wherein the antibody or antigen-binding fragment thereof blocks the interaction between human LILRB2 and human ANGPTL1;
the antibody or antigen-binding fragment thereof blocks the interaction between human LILRB2 and human ANGPTL2;
the antibody or antigen-binding fragment thereof blocks the interaction between human LILRB2 and human ANGPTL4;
the antibody or antigen-binding fragment thereof blocks the interaction between human LILRB2 and human ANGPTL7; or
the antibody or antigen-binding fragment thereof blocks the interaction between human LILRB2 and human MAG.

13. (canceled)

14. (canceled)

15. (canceled)

16. (canceled)

17. The antibody or antigen-binding fragment thereof of claim 1, wherein the antibody or antigen-binding fragment thereof is capable to reprogram human monocyte derived M2 macrophages into M1 phenotype;
the antibody or antigen-binding fragment thereof relieves M2 macrophages mediated suppression of T cells;
the antibody or antigen-binding fragment thereof promotes human dendritic cells into a maturation status;
the antibody or antigen-binding fragment thereof promotes human monocytes into a pro-inflammation status;
the antibody or antigen-binding fragment thereof promotes human PBMCs into a pro-inflammation status;
the antibody or antigen-binding fragment thereof upregulate the release of pro-inflammatory cytokines and/or chemokines;
optionally,
the pro-inflammatory cytokines and/or chemokines are selected from the group consisting of IL-2, IFN-γ, TNF-α, Granzyme B, PDGF-AA, IL-3, IL-1beta, IL-5, TGF-alpha, IFN-alpha, CCL4, FGF-basic, IL-13, G-CSF, IL-4, PD-L1, CCL20, IL-15, GM-CSF, CCL5, CCL3, PDGF-AB, CCL19, CXCL2, IL-6, VEGF, and the combination thereof; or
the antibody or antigen-binding fragment thereof reduces the expression of IL-10.

18. (canceled)

19. (canceled)

20. (canceled)

21. (canceled)

22. (canceled)

23. (canceled)

24. (canceled)

25. (canceled)

26. The antibody or antigen-binding fragment thereof of claim 1, wherein the antibody or antigen-binding fragment thereof is a chimeric or humanized antibody or antigen-binding fragment thereof;
optionally, the antibody or antigen-binding fragment thereof is an IgG1 antibody, IgG2 antibody, IgG3 antibody, or IgG4 antibody or antigen-binding fragment thereof.

27. (canceled)

28. An antibody or antigen-binding fragment thereof which binds to the peptide including amino acid sequence set forth in SEQ ID NO: 21.

29. An isolated polynucleotide encoding the antibody or antigen-binding fragment thereof of claim 1.

30. An isolated vector comprising the polynucleotide according to claim 29.

31. A host cell comprising the isolated vector of claim 30.

32. A pharmaceutical composition comprising the antibody or antigen-binding fragment thereof of claim 1, and a pharmaceutically acceptable carrier.

33. A kit comprising the antibody or antigen-binding fragment thereof of claim 1.

34. A pharmaceutical composition comprising the antibody or antigen-binding fragment thereof of claim 1 and an anti-PD-1 antibody.

35. (canceled)

36. (canceled)

37. A method of manufacture, comprising:
using the antibody or antigen-binding fragment thereof of claim 1 in the manufacture of a therapeutic agent for enhancing immune response,
optionally, the therapeutic agent binds to human LILRB2, or
therapeutic agent block is the interaction between human LILRB2 and HLA-A2, HLA-G, human ANGPTL1, human ANGPTL2, human ANGPTL4, human ANGPTL7, and/or human MAG.

38. A method of manufacture, comprising:
using the antibody or antigen-binding fragment thereof of claim 1 in the manufacture of a therapeutic agent for diagnosing, preventing or treating a tumor;
optionally, at least a tumor cell expressing LILRB2.

39. (canceled)

40. A method of enhancing immune response in a subject in need thereof, comprising administrating to the subject a therapeutically effective amount of the antibody or antigen-binding fragment thereof of claim 1;
optionally,
the antibody or antigen-binding fragment thereof reprograms human monocyte derived M2 macrophages into M1 phenotype,
the antibody or antigen-binding fragment thereof relieves tumor-associated macrophages M2 phenotype mediated suppression of T cells,
the antibody or antigen-binding fragment thereof promotes human dendritic cells into a maturation status,
the antibody or antigen-binding fragment thereof promotes human monocytes into a pro-inflammation status,
the antibody or antigen-binding fragment thereof promotes human PBMCs into a pro-inflammation status;
the antibody or antigen-binding fragment thereof upregulate the release of pro-inflammatory cytokines and/or chemokines, the said pro-inflammatory cytokines and/or chemokines are selected from the group consisting of IL-2, IFN-γ, TNF-α, Granzyme B, PDGF-AA, IL-3, IL-1beta, IL-5, TGF-alpha, IFN-alpha, CCL4, FGF-basic, IL-13, G-CSF, IL-4, PD-L1, CCL20, IL-15, GM-CSE, CCL5, CCL3, PDGF-AB, CCL19, CXCL2, IL-6, VEGF, and the combination thereof; and/or
the antibody or antigen-binding fragment thereof reduces the expression of IL-10.

41. (canceled)

42. (canceled)

43. (canceled)

44. A method of diagnosing, preventing or treating a disease, disorder or condition in a subject in need thereof, comprising administrating to the subject a therapeutically effective amount of the antibody or antigen-binding fragment thereof of claim 1;
the disease, disorder or condition comprises a tumor, optionally, at least a tumor cell expresses LILRB2;
optionally,
the subject is mammals including a human.

45. (canceled)

46. (canceled)

47. A binding epitope on LILRB2, wherein the binding epitope comprises the amino acid sequence set forth in SEQ ID NO: 21, optionally, the LILRB2 is human LILRB2.

48. (canceled)

49. The method of claim 37, wherein the interaction between human LILRB2 and HLA-A2, HLA-G, human ANGPTL1, human ANGPTL2, human ANGPTL4, human ANGPTL7, or human MAG is blocked.
